(19) United States
(12) Patent Application Publication
(10) Pub. No.: US 2008/0279896 A1
(43) Pub. Date: Nov. 13, 2008

(54) Treatment of movement disorders by a combined use of chemodenervating agent and automated movement therapy

(75) Inventors: Florian Heinen, Munchen (DE); Susanne Grafe, Frankfurt (DE); Steffen Berweck, Munchen (DE); Ingo Borggraefe, Munchen (DE)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(73) Assignee: MERZ PHARMA GMBH & CO., KGAA, FRANKFURT AM MAIN (DE)

(21) Appl. No.: 12/150,161

(22) Filed: Apr. 25, 2008

Related U.S. Application Data
(60) Provisional application No. 60/926,305, filed on Apr. 26, 2007.

(30) Foreign Application Priority Data
Apr. 26, 2007 (EP) 07008528.7

(51) Int. Cl. A61K 39/08

(52) U.S. Cl. 424/239.1

(57) ABSTRACT

The present invention relates to a method of treating a movement disorder in a patient, the method comprising administering a medicament comprising an effective amount of chemodenervating agent to the patient, wherein the patient is subjected to a muscle stimulation therapy, for example an movement therapy or an muscle activation therapy, and the medicament is administered prior to and/or during and/or after the movement therapy and a kit for the treatment of patients suffering from movement disorders comprising a medicament comprising an effective amount of a chemodenervating agent, and a device for carrying out automated movement therapy.

### FIELD OF THE INVENTION

The present invention relates to methods for treating movement disorders by a combined use of a chemodenervating agent and automated muscle stimulation therapy and a kit comprising the chemodenervating medicament and a device for performing an, optionally automated, muscle stimulation e.g. movement therapy.

### BACKGROUND OF THE INVENTION

A. Chemodenervating Agents

Chemodenervation refers to the use of an agent to prevent a nerve from stimulating its target tissue, e.g. a muscle, a gland or another nerve. Chemodenervation is for example performed with phenol, ethyl alcohol, or botulinum toxin. Chemodenervation is for example appropriate in patients with localized spasticity in one or two large muscles or several small muscles. It may be used to alleviate symptoms such as muscle spasm and pain, and hyperreflexia. Chemodenervating agents capable of interfering with muscle innovation may also be called “muscle relaxant”.

The term “muscle relaxant” is used herein to refer to at least two major therapeutic groups: neuromuscular blockers and spasmolytics. Neuromuscular blockers act by interfering with transmission at the neuromuscular end plate and have no CNS activity. They are often used during surgical procedures, in intensive care and emergency medicine to cause partly or complete paralysis or dose dependent paresis, respectively (i.e. are used as an modulator of muscle tonus). Spasmolytics, also known as “centrally-acting” muscle relaxants, are used to alleviate musculoskeletal pain and spasms and to reduce spasticity in a variety of neurological conditions. Neuromuscular blockers and spasmolytics are often grouped together as muscle relaxants. both terms refer to distinct groups of agents.

Neuromuscular-blocking drugs block neuromuscular transmission at the neuromuscular junction, causing paralysis or paresis of the affected skeletal muscles. This is accomplished either by acting presynaptically via the inhibition of acetylcholine (ACh) synthesis or release, or by acting post-synaptically at the acetylcholine receptor. Example of drugs that act pre-synaptically are botulinum toxin, tetrodotoxin and tetanus toxin.

The term “chemodenervation” also encompasses all effects which directly or indirectly are induced by the chemodenervating agent, therefore also comprising upstream, downstream or long-term effects of said chemodenervating agent. Therefore presynaptic effects are also encompassed as well as postsynaptic effects, tissue effects and/or indirect effects via spinal or afferent neurons.

One chemodenervating agent, botulinum toxin, although being one of the most toxic compounds known to date, has in the past been used for the treatment of a large number of conditions and disorders, some of which are described in e.g. PCT/EP 2007/005754. Furthermore, commercial forms of botulinum toxin type A based on the botulinum toxin A protein complex are available under the tradename Botox® (Allergan Inc.) and under the tradename Dysport® (Ipsen Ltd.), respectively. A pharmaceutical composition based on a higher purified toxin preparation and comprising the neurotoxic component of botulinum toxin type A free of complexing proteins in isolated form is commercially available in Germany from Merz Pharmaceuticals GmbH under the tradename Xeomin®.

### Muscle Stimulation Therapy

Various muscle stimulation therapies are known in the art. In this regard we refer to the subsections B of the “Detailed Description of the Invention” herein.

One particular embodiment is the locomotion therapy.

Locomotion therapy for regaining walking capacity using the principle of enhancing neuroplasticity by task specific training has been well established in the (re)habilitation process of patients with central gait disorders (see e.g. Hesse S. (2001) Locomotor therapy in neurorehabilitation, NeuroRehabilitation 16: 133-139).; Borggraefe et al (2007) Movement Disorders, 23, 280-282; Meyer-Heim et al (2007) Developmental Medicine & Child Neurology 2007, 49, 900, 906.

Conventional over-ground gait training (COGT) in adults has been endorsed by the method body weight supported treadmill training (BWSTT), thereby gaining functional benefits such as symmetry and increased walking speed (Barbeau H, Visintin M. (2003) Optimal outcomes obtained with body-weight support combined with treadmill training in stroke subjects, Arch Phys Med Rehabil 84: 1458-1465; McNevin N H, Coraci L, Schafer J. (2000) Gait in adolescent cerebral palsy: the effect of partial unweighting, Arch Phys Med Rehabil 81: 525-528). However, the assignment of human resources for manual assistance in these methods is considerable. Controlled trials of adult patients with traumatic brain injury (TBI) or incomplete spinal cord injury (SCI) have been conducted by using BWSTT and COGT (Dobkin B, Apple D, Barbeau H, Basso M, Behrman A, Deforge D, et al. (2006) Weight-supported treadmill vs over-ground training for walking after acute incomplete SCI, Neurology 66: 484-493; Wilson D J, Powell M, Gorham J L, Childers M K (2006) Ambulation training with and without partial weightbearing after traumatic brain injury: results of a randomized, controlled trial, Am J Phys Med Rehabil 85: 68-74).

In children with cerebral palsy (CP), general functional-strength training has been found effective to improve functional performance. Promising evidence exists that intensive BWSTT can improve walking capacity in these children (Song W H S I, Kim Y J, Yoo J Y, (2003) Treadmill training with partial body weight support in children with cerebral palsy, Arch Phys Med Rehabil 84 (E2)).

In order to restore or develop walking abilities, repetition and intensity of training seems to be a crucial key to motor (re)learning. Thus, automated gait training devices have been developed during the last decade to further improve gait training (Colombo G, Joerg M, Schreier R, Dietz V. (2000) Treadmill training of paraplegic patients using a robotic orthosis, J Rehabil Res Dev 37: 693-700; Hesse S, Schmidt H, Werner C, Bardeleben A (2003), Upper and lower extremity robotic devices for rehabilitation and for studying motor control, Curr Opin Neurol 16: 705-710).

Such a device is described in detail in U.S. Pat. No. 6,821,233. The patent relates to an automatic machine to be used in treadmill therapy of paraparetic and hemiparetic patients and which automatically guides the legs on the treadmill. The machine of the invention consists of a driven and controlled orthotic device which guides the legs in a physiological pattern of movement, a treadmill and a relief mechanism. An improved relief mechanism, which helps to support the patient by unloading at least some of its body weight is the subject of EP 1 586 291. Further devices for locomotion therapy are the subject of U.S. Pat. No. 6,059,506 and U.S. Pat. No. 6,685,658, respectively.

Devices for automated locomotion therapy are commercially available from Hocoma A G, e.g. under the trademark Lokomat®. A pediatric module of the DGO Lokomat® has been developed very recently, which allows training of children starting at an age of approximately 4 years and older.

One specific aspect of the movement disorders that are to be treated according to the present invention is related to cerebral palsy (CP) in children. CP is the most frequent movement disorder in children. It occurs within 1.5 to 2.5 per 1000 children. CP is a disorder of the development of movement and appearance and is caused by damages within the young brain still to be developed (early brain lesion). As such, CP is a collective name given to a range of conditions caused by, e.g., early brain lesion caused before, at or around the time of birth, or in the first year of life. As used herein, CP also includes any other cause for diseases or disorders resulting in hyperactive muscles. The brain injury may be caused by a variety of conditions, e.g. by prematurity. Although the brain injury causing cerebral palsy is a non-progressive injury, its effects may change as the patient grows older. This may result in dynamic contractures of the muscles, which tend to change over time to fixed contractures and which impair or inhibit completely the patient's ability to use the affected muscles.

Besides the above-mentioned locomotion therapy, the method of choice for treating CP, in many cases traditionally was surgery. In recent years, botulinum toxin as a bacterial toxin has been used in the treatment of cerebral palsy. An overview of the treatment of CP with botulinum toxin may be found in European patent EP 0 605 501 as well as in the “European Consensus Table 2006 on botulinum toxin for children with cerebral palsy”, European Journal of Pediatric Neurology 10 (2006), 215-225 and the literature cited therein.

### OBJECTS OF THE INVENTION

In view of the above-cited prior art, it is an object of the present invention to provide an alternative treatment of movement disorders, e.g. for the treatment of movement disorders occurring in conjunction with CP in children. It is a further object of the present invention to provide an improved therapy of these disorders that is in one embodiment related to individual motor development.

It is still another object to provide a kit specifically designed for a patient suffering from movement disorders of the kind to be treated herein.

### SUMMARY OF THE INVENTION

What we therefore believe to be comprised by our invention may be summarized inter alia in the following words:

A chemodenervating agent which is administered to a patient to treat a movement disorder in the patient, wherein the patient is a patient who is, has been and/or will be subjected to a muscle stimulation therapy, and wherein the chemodenervating agent is administered prior to and/or during and/or after the muscle stimulation therapy, such a

chemodenervating agent, wherein the muscle stimulation therapy is an automated muscle stimulation therapy, such a

chemodenervating agent, wherein the muscle stimulation therapy is a muscle activation therapy, wherein the muscle activation refers to an elevation of muscle metabolism above resting state of the muscle, such a

chemodenervating agent, wherein the muscle stimulation therapy is an automated movement therapy, such a

chemodenervating agent, wherein the muscle activation therapy is temperature stimulation, electric stimulation, vibration, activation by sound-waves, activation by hydrostatic means, activation by electro-magnetic waves or magnetic fields, pharmaceutical activation or any combination thereof, such a

chemodenervating agent, wherein the temperature stimulation is a heating of the target muscle above 40°, or above 45° C., or above 50° C., up to 55° C., up to 60° C., up to 70° C. or up to 80° C., such a chemodenervating agent, wherein the automated muscle activation by temperature stimulation is a cooling of the target muscle to below 35° C., or below 30° C., or below 25° C., or below 20° C., or below 10° C., down to 0° C., down to −5° C., down to −10° C. or down to −20° C., such a

chemodenervating agent, wherein the electric stimulation is directed to the nerves innervating the target muscle, such a

chemodenervating agent, wherein the electric stimulation is directed to the target muscle itself, such a

chemodenervating agent, wherein the vibration is directed to the whole body, such a

chemodenervating agent, wherein the vibration is directed to a single muscle, muscle group or limb, such a

chemodenervating agent, wherein the sound-waves are ultrasound waves or acoustical waves, such a

chemodenervating agent, wherein the hydrostatic means comprise water-jets, such a

chemodenervating agent, wherein the electro-magnetic waves comprise microwaves, such a

chemodenervating agent, wherein the magnetic fields comprise magnetic stimulation, such a

chemodenervating agent, wherein the pharmaceutical activation comprises the administration of a stimulant, a muscle contractant, a substance which increases blood flow within the muscle, a substance which raises the muscle temperature, a substance which up-regulates the number of surface proteins thereby allowing the chemodenervating agent to bind and enter the cell or any combination thereof, such a

chemodenervating agent, wherein the stimulant is selected from the group of a β₃ agonist, caffeine, ephedrine, amphetamine, methamphetamine, methylphenidate, cocaine-derivate and any combination thereof, such a

chemodenervating agent, wherein the muscle contractant is selected from the group of a substance with sympathetic effect, a substance with agonistic effects on β₂-adrenergic receptor, caffeine, acetylcholine, nicotine, epibatidine-derivatives, ABT-594, dimethylphenylpiperazinium, succinyl choline, a muscle stimulating saponin-derivative isolated from Dalbergia saxatilis, calcium, potassium, norepinephrine, adrenaline (epinephrine), leukotrienes, allene containing arachidonic acid derivatives and any combination thereof, such a

chemodenervating agent, wherein the substance which increases blood flow within the muscle is selected from the group of EDHF, interstitial K⁺, nitric oxide, β₂ adrenergic agonists, histamine, prostacyclin, prostaglandin, VIP, extracellular adenosine, extracellular ATP, extracellular ADP, L-Arginine, bradykinin, substance P, niacin (nicotinic acid), platelet activating factor (PAF), CO₂, interstitial lactic acid, Adenocard®, alpha blockers, amyl nitrite, atrial natriuretic peptide, ethanol, histamine-inducers, complement proteins C3a, C4a, C5a, nitric oxide inducers, glyceryl trinitrate (nitroglycerin), isosorbide mononitrate, isosorbide dinitrate, pentaerythritol tetranitrate (PETN), sodium nitroprusside, PDE5 inhibitors, agents which indirectly increase the effects of nitric oxide, sildenafil, tadalafil, tardenafil, tetrahydrocannabinol, theobromine, papaverine and any combination thereof, such a

chemodenervating agent, wherein the substance which raises the muscle temperature is selected from the group of ephedra, bitter orange (synephrine), capsicum, ginger, sibutramine and its metabolites, caffeine and any combination thereof, such a

chemodenervating agent, wherein the surface protein is selected from the group comprising a substance which up-regulates SV2, GT1b, GD1b, GQ1b, synaptotagmin polypeptides, Syt1 and Syt2, such a

chemodenervating agent, wherein the substance which up-regulates the number of surface proteins is selected from the group comprising hormones, growth factors, neurotrophins, blocking substances of receptor-internalization, factors which enhance the receptor surface expression, arrestin-inhibitors, protease inhibitors, blocking substances of receptor degradation, inhibitors of inhibitory G-proteins, competitive receptor antagonists and neurotransmitter degrading agents, such a

chemodenervating agent, wherein the automated movement therapy is supported by an automated gait orthosis or an arm mover, such a

chemodenervating agent, wherein the automated gait orthosis is used in combination with a treadmill, such a

chemodenervating agent, wherein the automated movement therapy is carried out by using a device comprising a driven and controlled orthotetic device which guides the legs of the patient in a physiological pattern of movement, in one embodiment using a treadmill and a relief mechanism acting on the body weight of the patient, such a

chemodenervating agent, wherein the relief mechanism comprises means for adjusting the height of and the relief force acting on the weight of the patient, wherein the weight is supported by a cable, with a first cable length adjustment means to provide an adjustment of the length of the cable to define the height of the suspended weight and a second cable length adjustment means to provide an adjustment of the length of the cable to define the relief force acting on the suspended weight, such a

chemodenervating agent, wherein the automated movement therapy is carried out by employing an apparatus for treadmill training, comprising a treadmill, a relief mechanism for the patient, and a driven orthotic device, wherein a parallelogram fixed in a height-adjustable manner on the treadmill is provided for stabilizing the orthotic device and preventing the patient from tipping forward, backwards and sidewards, the parallelogram being attached to the orthotic device; the orthotic device comprises a hip orthotic device and two leg parts, whereby two hip drives are provided for moving the hip orthotic device, and two knee drives are provided for moving the leg parts; the hip orthotic device and leg parts are adjustable, the leg parts are provided with cuffs which are adjustable in size and position; a control unit is provided for controlling the movements of the orthotic device and controlling the speed of the treadmill, such a

chemodenervating agent, wherein the automated movement therapy is carried out by employing an apparatus for treadmill training, comprising a treadmill including a railing, a relief mechanism for the patient, and a driven orthotic device, wherein means for stabilizing the orthotic device are provided that prevent the patient from tipping forward, backward and sideward; the orthotic device comprises a hip orthotic device and two leg parts, two hip drives are provided for moving the hip orthotic device, and two knee drives are provided for moving the leg parts; a ball screw spindle drive is provided for each knee drive and hip drive, the orthotic device and leg parts are adjustable, the leg parts are provided with cuffs which are adjustable in size and position; a control unit is provided for controlling the movements of the orthotic device and controlling the speed of the treadmill, such a

chemodenervating agent, wherein the automated movement therapy is carried out by employing an apparatus for locomotion therapy for the rehabilitation or habilitation of bilateral or unilateral spastic conditions in paraparetic and hemiparetic patients, comprising a standing table adjustable in height and inclination, a fastening belt with holding devices on the standing table for the patient, a drive mechanism for the leg movement of the patient, consisting of a knee mechanism and a foot mechanism, wherein the standing table has a head portion displaceable with respect to a leg portion about a pivot joint, whereby the pivot joint provides an adjustable hip extension angle for which an adjusting mechanism is provided; and the knee portion and foot portion are displaceably arranged on rails on the leg mechanism; the foot mechanism serves to establish force on the sole of the foot during knee extension; a control unit is provided for controlling movement of the apparatus, such a

chemodenervating agent, wherein the automated movement therapy is carried out by employing a device for applying a force between first and second portions of an animate body, the device comprising: first and second link assemblies associated with the first and second portions, respectively, each the first and second link assembly comprising:
A. a supporting section secured in position on a portion, each supporting section being a supporting link; and
B. an articulated link attached through a joint to each of the supporting links; wherein the articulated links of the first and second link assemblies are attached to each other through a pivot joint, with the articulated link of the second assembly extending beyond the pivot joint; first and second casings attached to a link in the first link assembly; first and second tendons extending through the first and second casings, respectively, and attached to a link in the second link assembly, wherein one of the tendons is attached to the articulated link in the second link assembly on one side of the pivot joint and the other tendon is attached to the articulated link in the second link assembly on the opposite side of the pivot joint, such a

chemodenervating agent, wherein the automated movement therapy is carried out by employing a device for applying a force between first and second portions of a hand, one of the portions being a phalanx, the device comprising: first and second link assemblies associated with the first and second portions, respectively, each link assembly comprising:
A. a supporting section secured in position on a portion, each supporting section being a supporting link; and
B. an articulated link attached through a joint to each of the supporting links; wherein the articulated links of the first and second link assemblies are attached to each other through a pivot joint, with the articulated link of the second assembly extending beyond the pivot joint; first and second casings attached to a link in the first link assembly; and first and second tendons extending through the first and second casings, respectively, and attached to a link in the second link assembly, wherein one of the tendons is attached to the articulated link in the second link assembly on one side of the pivot joint and the other tendon is attached to the articulated link in the second assembly on the opposite side of the pivot joint.

chemodenervating agent, wherein the movement disorder is a hyperkinetic and/or hypokinetic movement disorder, wherein an imbalance between agonist and antagonist is interfering with function, such a

chemodenervating agent, wherein the movement disorder is associated with cerebral palsy, M. Parkinson, central gait impairment, spinal cord injuries, dystonias, traumatic brain injury, genetic disorders, metabolic disorders, dynamic muscle contractures and/or stroke, such a

chemodenervating agent, wherein the movement disorder is associated with at least one selected among pes equinus, pes varus, lower limb spasticity, upper limb spasticity, adductor spasticity, hip flexion contracture, hip adduction, knee flexion spasticity (crouch gait), plantar flexion of the ankle, supination and pronation of the subtalar joint, writer's cramp, musician's cramp, golfer's cramp, leg dystonia, thigh adduction, thigh abduction, knee flexion, knee extention, equinovarus deformity, foot dystonia, striatal toe, toe flexion, toe extension, such a

chemodenervating agent, wherein the patient is human, such a

chemodenervating agent, wherein the patient has not completed its motor development and fixed muscle contractures have not occurred, such a

chemodenervating agent, wherein the patient is a child up to six years in age, such a

chemodenervating agent, wherein the chemodenervating agent is a botulinum toxin, such a

chemodenervating agent which is administered by injection, such a

chemodenervating agent which is administered several times during the treatment, such a

chemodenervating agent which is administered for the first time before commencement of the movement therapy, such a

chemodenervating agent which is re-administered in intervals of between 3 and 6 months, such a

chemodenervating agent which is re-administered in intervals of between 2 weeks and less than 3 months, such a

chemodenervating agent which is re-administered at a point in time when muscular activity interferes with the automated muscle activation therapy, such a

chemodenervating agent, wherein the effective amount of botulinum toxin administered exceeds 500 U of neurotoxic component in adults or exceeds 15 U/kg body weight in children, such a

chemodenervating agent, wherein the botulinum toxin is the botulinum toxin complex type A, such a

chemodenervating agent, wherein the botulinum toxin is the neurotoxic component of a Clostridium botulinum toxin complex, such a

chemodenervating agent, wherein the botulinum toxin is selected from the group consisting of serotypes A, B, C, D, E, F, G and a mixture thereof, such a

chemodenervating agent, wherein the neurotoxic component is of type A, such a

method of treating a movement disorder in a patient, the method comprising administering a medicament comprising an effective amount of a chemodenervating agent to the patient, wherein the patient is a patient who is, has been and/or will be subjected to a muscle stimulation therapy, and wherein the chemodenervating agent is administered prior to and/or during and/or after the muscle stimulation therapy, such a

method wherein the muscle stimulation therapy is an automated muscle stimulation therapy, such a

method wherein the muscle stimulation therapy is a muscle activation therapy, wherein the muscle activation refers to an elevation of muscle metabolism above resting state of the muscle, such a

method wherein the muscle stimulation therapy is an automated movement therapy, such a

method wherein the muscle activation therapy is temperature stimulation, electric stimulation, vibration, activation by sound-waves, activation by hydrostatic means, activation by electro-magnetic waves or magnetic fields, pharmaceutical activation or any combination thereof, such a

method wherein the temperature stimulation is a heating of the target muscle above 40°, or above 45° C., or above 50° C., up to 55° C., up to 60° C., up to 70° C. or up to 80° C., such a

method wherein the automated muscle activation by temperature stimulation is a cooling of the target muscle to below 35° C., or below 30° C., or below 25° C., or below 20° C., or below 10° C., down to 0° C., down to −5° C., down to −10° C. or down to −20° C., such a

method wherein the electric stimulation is directed to the nerves innervating the target muscle, such a

method wherein the electric stimulation is directed to the target muscle itself, such a

method wherein the vibration is directed to the whole body, such a

method wherein the vibration is directed to a single muscle, muscle group or limb, such a

method wherein the sound-waves are ultrasound waves or acoustical waves, such a

method wherein the hydrostatic means comprise water-jets, such a

method wherein the electro-magnetic waves comprise microwaves, such a

method wherein the magnetic fields comprise magnetic stimulation, such a

method wherein the pharmaceutical activation comprises the administration of a stimulant, a muscle contractant, a substance which increases blood flow within the muscle, a substance which raises the muscle temperature, a substance which up-regulates the number of surface proteins thereby allowing the chemodenervating agent to bind and enter the cell or any combination thereof, such a

method wherein the stimulant is selected from the group of a β₃ agonist, caffeine, ephedrine, amphetamine, methamphetamine, methylphenidate, cocaine-derivate and any combination thereof, such a

method wherein the muscle contractant is selected from the group of a substance with sympathetic effect, a substance with agonistic effects on β₂-adrenergic receptor, caffeine, acetylcholine, nicotine, epibatidine-derivatives, ABT-594, dimethylphenylpiperazinium, succinyl choline, a muscle stimulating saponin-derivative isolated from Dalbergia saxatilis, calcium, potassium, norepinephrine, adrenaline (epinephrine), leukotrienes, allene containing arachidonic acid derivatives and any combination thereof, such a

method wherein the substance which increases blood flow within the muscle is selected from the group of EDHF, interstitial K⁺, nitric oxide, β₂ adrenergic agonists, histamine, prostacyclin, prostaglandin, VIP, extracellular adenosine, extracellular ATP, extracellular ADP, L-Arginine, bradykinin, substance P, niacin (nicotinic acid), platelet activating factor (PAF), CO₂, interstitial lactic acid, Adenocard®, alpha blockers, amyl nitrite, atrial natriuretic peptide, ethanol, histamine-inducers, complement proteins C3a, C4a, C5a, nitric oxide inducers, glyceryl trinitrate (nitroglycerin), isosorbide mononitrate, isosorbide dinitrate, pentaerythritol tetranitrate (PETN), sodium nitroprusside, PDE5 inhibitors, agents which indirectly increase the effects of nitric oxide, sildenafil, tadalafil, tardenafil, tetrahydrocannabinol, theobromine, papaverine and any combination thereof, such a

method wherein the substance which raises the muscle temperature is selected from the group of ephedra, bitter orange (synephrine), capsicum, ginger, sibutramine and its metabolites, caffeine and any combination thereof, such a

method wherein the surface protein is selected from the group comprising a substance which up-regulates SV2, GT1b, GD1b, GQ1b, synaptotagmin polypeptides, Syt1 and Syt2, such a

method wherein the substance which up-regulates the number of surface proteins is selected from the group comprising hormones, growth factors, neurotrophins, blocking substances of receptor-internalization, factors which enhance the receptor surface expression, arrestin-inhibitors, protease inhibitors, blocking substances of receptor degradation, inhibitors of inhibitory G-proteins, competitive receptor antagonists and neurotransmitter degrading agents, such a

method wherein the automated movement therapy is supported by an automated gait orthosis or an arm mover, such a

method wherein the automated gait orthosis is used in combination with a treadmill, such a

method wherein the automated movement therapy is carried out by using a device comprising a driven and controlled orthotetic device which guides the legs of the patient in a physiological pattern of movement, in one embodiment using a treadmill and a relief mechanism acting on the body weight of the patient, such a

method wherein the relief mechanism comprises means for adjusting the height of and the relief force acting on the weight of the patient, wherein the weight is supported by a cable, with a first cable length adjustment means to provide an adjustment of the length of the cable to define the height of the suspended weight and a second cable length adjustment means to provide an adjustment of the length of the cable to define the relief force acting on the suspended weight, such a

method wherein the automated movement therapy is carried out by employing an apparatus for treadmill training, comprising a treadmill, a relief mechanism for the patient, and a driven orthotic device, wherein a parallelogram fixed in a height-adjustable manner on the treadmill is provided for stabilizing the orthotic device and preventing the patient from tipping forward, backwards and sidewards, the parallelogram being attached to the orthotic device; the orthotic device comprises a hip orthotic device and two leg parts, whereby two hip drives are provided for moving the hip orthotic device, and two knee drives are provided for moving the leg parts; the hip orthotic device and leg parts are adjustable, the leg parts are provided with cuffs which are adjustable in size and position; a control unit is provided for controlling the movements of the orthotic device and controlling the speed of the treadmill, such a

method wherein the automated movement therapy is carried out by employing an apparatus for treadmill training, comprising a treadmill including a railing, a relief mechanism for the patient, and a driven orthotic device, wherein means for stabilizing the orthotic device are provided that prevent the patient from tipping forward, backward and sideward; the orthotic device comprises a hip orthotic device and two leg parts, two hip drives are provided for moving the hip orthotic device, and two knee drives are provided for moving the leg parts; a ball screw spindle drive is provided for each knee drive and hip drive, the orthotic device and leg parts are adjustable, the leg parts are provided with cuffs which are adjustable in size and position; a control unit is provided for controlling the movements of the orthotic device and controlling the speed of the treadmill, such a

method wherein the automated movement therapy is carried out by employing an apparatus for locomotion therapy for the rehabilitation or habilitation of bilateral or unilateral spastic conditions in paraparetic and hemiparetic patients, comprising a standing table adjustable in height and inclination, a fastening belt with holding devices on the standing table for the patient, a drive mechanism for the leg movement of the patient, consisting of a knee mechanism and a foot mechanism, wherein the standing table has a head portion displaceable with respect to a leg portion about a pivot joint, whereby the pivot joint provides an adjustable hip extension angle for which an adjusting mechanism is provided; and the knee portion and foot portion are displaceably arranged on rails on the leg mechanism; the foot mechanism serves to establish force on the sole of the foot during knee extension; a control unit is provided for controlling movement of the apparatus, such a

method wherein the automated movement therapy is carried out by employing a device for applying a force between first and second portions of an animate body, the device comprising: first and second link assemblies associated with the first and second portions, respectively, each the first and second link assembly comprising:
A. a supporting section secured in position on a portion, each supporting section being a supporting link; and
B. an articulated link attached through a joint to each of the supporting links; wherein the articulated links of the first and second link assemblies are attached to each other through a pivot joint, with the articulated link of the second assembly extending beyond the pivot joint; first and second casings attached to a link in the first link assembly; first and second tendons extending through the first and second casings, respectively, and attached to a link in the second link assembly, wherein one of the tendons is attached to the articulated link in the second link assembly on one side of the pivot joint and the other tendon is attached to the articulated link in the second link assembly on the opposite side of the pivot joint, such a

method wherein the automated movement therapy is carried out by employing a device for applying a force between first and second portions of a hand, one of the portions being a phalanx, the device comprising: first and second link assemblies associated with the first and second portions, respectively, each link assembly comprising:
A. a supporting section secured in position on a portion, each supporting section being a supporting link; and
B. an articulated link attached through a joint to each of the supporting links; wherein the articulated links of the first and second link assemblies are attached to each other through a pivot joint, with the articulated link of the second assembly extending beyond the pivot joint; first and second casings attached to a link in the first link assembly; and first and second tendons extending through the first and second casings, respectively, and attached to a link in the second link assembly, wherein one of the tendons is attached to the articulated link in the second link assembly on one side of the pivot joint and the other tendon is attached to the articulated link in the second assembly on the opposite side of the pivot joint, such a method wherein the movement disorder is a hyperkinetic and/or hypokinetic movement disorder, wherein an imbalance between agonist and antagonist is interfering with function, such a

method wherein the movement disorder is associated with cerebral palsy, M. Parkinson, central gait impairment, spinal cord injuries, dystonias, traumatic brain injury, genetic disorders, metabolic disorders, dynamic muscle contractures and/or stroke, such a

method wherein the movement disorder is associated with at least one selected among pes equinus, pes varus, lower limb spasticity, upper limb spasticity, adductor spasticity, hip flexion contracture, hip adduction, knee flexion spasticity (crouch gait), plantar flexion of the ankle, supination and pronation of the subtalar joint, writer's cramp, musician's cramp, golfer's cramp, leg dystonia, thigh adduction, thigh abduction, knee flexion, knee extention, equinovarus deformity, foot dystonia, striatal toe, toe flexion, toe extension, such a

method wherein the patient is human, such a

method wherein the patient has not completed its motor development and fixed muscle contractures have not occurred, such a

method wherein the patient is a child up to six years in age, such a

method wherein the chemodenervating agent is a botulinum toxin, such a

method which is administered by injection, such a

method which is administered several times during the treatment, such a

method which is administered for the first time before commencement of the movement therapy, such a

method which is re-administered in intervals of between 3 and 6 months, such a

method which is re-administered in intervals of between 2 weeks and less than 3 months, such a

method which is re-administered at a point in time when muscular activity interferes with the automated muscle activation therapy, such a

method wherein the effective amount of botulinum toxin administered exceeds 500 U of neurotoxic component in adults or exceeds 15 U/kg body weight in children, such a

method wherein the botulinum toxin is the botulinum toxin complex type A, such a

method wherein the botulinum toxin is the neurotoxic component of a Clostridium botulinum toxin complex, such a

method wherein the botulinum toxin is selected from the group consisting of serotypes A, B, C, D, E, F, G and a mixture thereof, such a

method wherein the neurotoxic component is of type A, such a

a kit for the treatment of patients suffering from movement disorders comprising,
a) a medicament comprising an effective amount of a chemodenervating agent; and
b) means for carrying out a muscle stimulation therapy, such a

a kit wherein the means for carrying out the muscle stimulation therapy is selected from the group of temperature stimulation means, electric stimulation means, vibration means, activation by sound-wave means, hydrostatic means, electro-magnetic wave means and magnetic field means, or any combination thereof, such a

A kit wherein the means for carrying out the muscle stimulation therapy is an automated movement therapy comprising a driven and controlled gait orthosis and/or arm mover which guides the extremities of a patient in a physiological pattern of movement, such a

kit wherein the chemodenervating agent is a botulinum toxin, such a

a kit wherein the botulinum toxin is the neurotoxic component of a Clostridium botulinum toxin complex.

In one embodiment the above and other objects are solved by a medicament comprising an effective amount of a chemodenervating agent for administering to a patient for treating a movement disorder in said patient, wherein said patient is a patient who is, has been and/or will be subjected to a muscle stimulation therapy, and wherein said medicament is administered prior to and/or during and/or after said muscle stimulation therapy.

In another embodiment the above and other objects are solved by the use of an effective amount of a chemodenervating agent for the manufacture of a medicament for administering to a patient for treating a movement disorder in said patient, wherein said patient is a patient who is, has been and/or will be subjected to an automated muscle stimulation therapy, and wherein said medicament is administered prior to and/or during and/or after said muscle stimulation therapy.

Said muscle stimulation therapy is an automated muscle stimulation therapy. Said automated muscle stimulation therapy is a muscle activation therapy, wherein the muscle activation refers to an elevation of muscle metabolism above resting state of said muscle. In another embodiment said automated muscle stimulation therapy is a muscle movement therapy.

In one embodiment said muscle stimulation therapy is an automated movement therapy.

In a further embodiment said muscle activation temperature stimulation, electric stimulation, vibration, activation by sound-waves, activation by hydrostatic means, activation by electro-magnetic waves or magnetic fields, pharmaceutical activation or any combination thereof. In one embodiment said temperature stimulation is a heating of the target muscle above 400, or above 45° C., or above 50° C., up to 55° C., up to 60° C., up to 70° C. or up to 80° C.

In another embodiment said automated muscle activation by temperature stimulation is a cooling of the target muscle to below 35° C., or below 30° C., or below 25° C., or below 20° C., or below 10° C., down to 0° C., down to −5° C. down to −10° C. or down to −20° C. In another embodiment said electric stimulation is directed to the nerves innervating the target muscle.

In another embodiment said electric stimulation is directed to the target muscle itself.

In another embodiment said vibration is directed to the whole body. In another embodiment said vibration is directed to a single muscle, muscle group or limb. In another embodiment said sound-waves are ultrasound waves or acoustical waves. In another embodiment said ultrasound or acoustical waves are directed to a single muscle, muscle group or limb.

In another embodiment said hydrostatic means comprise water-jets. In another embodiment, the water jets are directed to a single muscle, muscle group or limb.

In another embodiment said electro-magnetic waves comprise microwaves. In another embodiment said electro-magnetic waves are directed to a single muscle, muscle group or limb.

In another embodiment, magnetic fields comprise magnetic stimulation.

In a further embodiment the pharmaceutical activation comprises the administration of a stimulant, a muscle contractant, a substance which increases blood flow within the muscle, a substance which raises the muscle temperature, a substance which up-regulates the number of surface proteins thereby allowing the chemodenervating agent to bind and enter the cell or any combination thereof.

In one embodiment said stimulant is selected from the group of a β₃ agonist, caffeine, ephedrine, amphetamine, methamphetamine, methylphenidate, cocaine-derivate and any combination thereof.

In another embodiment said muscle contractant is selected from the group of a substance with sympathetic effect, a substance with agonistic effects on β₂-adrenergic receptor, caffeine, acetylcholine, nicotine, epibatidine-derivatives, ABT-594, dimethylphenylpiperazinium, succinyl choline, a muscle stimulating saponin-derivative isolated from Dalbergia saxatilis, calcium, potassium, norepinephrine, adrenaline (epinephrine), leukotrienes, allene containing arachidonic acid derivatives and any combination thereof.

In another embodiment said substance which increases blood flow within the muscle is selected from the group of EDHF, interstitial K⁺, nitric oxide, β2 adrenergic agonists, histamine, prostacyclin, prostaglandin, VIP, extracellular adenosine, extracellular ATP, extracellular ADP, L-Arginine, bradykinin, substance P, niacin (nicotinic acid), platelet activating factor (PAF), CO₂, interstitial lactic acid, Adenocard®, alpha blockers, amyl nitrite, atrial natriuretic peptide, ethanol, histamine-inducers, complement proteins C3a, C4a, C5a, nitric oxide inducers, glyceryl trinitrate (nitroglycerin), isosorbide mononitrate, isosorbide dinitrate, pentaerythritol tetranitrate (PETN), sodium nitroprusside, PDE5 inhibitors, agents which indirectly increase the effects of nitric oxide, sildenafil, tadalafil, tardenafil, tetrahydrocannabinol, theobromine, papaverine and any combination thereof. In another embodiment said substance which raises the muscle temperature is selected from the group of ephedra, bitter orange (synephrine), capsicum, ginger, sibutramine and its metabolites, caffeine and any combination thereof.

In another embodiment said surface protein is selected from the group comprising a substance which up-regulates SV2, GT1b, GD1b, GQ1b, synaptotagmin polypeptides, Syt1 and Syt2.

In another embodiment said substance which up-regulates the number of surface proteins is selected from the group comprising hormones, growth factors, neurotrophins, blocking substances of receptor-internalization, factors which enhance the receptor surface expression, arrestin-inhibitors, protease inhibitors, blocking substances of receptor degradation, inhibitors of inhibitory G-proteins, competitive receptor antagonists and neurotransmitter degrading agents.

In yet another embodiment said automated movement therapy is supported by an automated gait orthosis or an arm mover.

In one embodiment said automated gait orthosis is used in combination with a treadmill.

In another embodiment said automated movement therapy is carried out by using a device comprising a driven and controlled orthotetic device which guides the legs of said patient in a physiological pattern of movement, in one embodiment using a treadmill and a relief mechanism acting on the body weight of said patient.

In another embodiment the relief mechanism comprises means for adjusting the height of and the relief force acting on the weight of the patient, wherein said weight is supported by a cable, with a first cable length adjustment means to provide an adjustment of the length of the cable to define the height of said suspended weight and a second cable length adjustment means to provide an adjustment of the length of the cable to define the relief force acting on the suspended weight.

In another embodiment wherein the automated movement therapy is carried out by employing an apparatus for treadmill training, comprising a treadmill, a relief mechanism for the patient, and a driven orthotic device, wherein a parallelogram fixed in a height-adjustable manner on the treadmill is provided for stabilizing the orthotic device and preventing the patient from tipping forward, backwards and sidewards, the parallelogram being attached to the orthotic device; the orthotic device comprises a hip orthotic device and two leg parts, whereby two hip drives are provided for moving the hip orthotic device, and two knee drives are provided for moving the leg parts; the hip orthotic device and leg parts are adjustable, the leg parts are provided with cuffs which are adjustable in size and position; a control unit is provided for controlling the movements of the orthotic device and controlling the speed of the treadmill.

In another embodiment the automated movement therapy is carried out by employing an apparatus for treadmill training, comprising a treadmill including a railing, a relief mechanism for the patient, and a driven orthotic device, wherein means for stabilizing the orthotic device are provided that prevent the patient from tipping forward, backward and sideward; the orthotic device comprises a hip orthotic device and two leg parts, two hip drives are provided for moving the hip orthotic device, and two knee drives are provided for moving the leg parts; a ball screw spindle drive is provided for each knee drive and hip drive, the orthotic device and leg parts are adjustable, the leg parts are provided with cuffs which are adjustable in size and position; a control unit is provided for controlling the movements of the orthotic device and controlling the speed of the treadmill.

In another embodiment the automated movement therapy is carried out by employing an apparatus for locomotion therapy for the rehabilitation or habilitation of bilateral or unilateral spastic conditions in paraparetic and hemiparetic patients, comprising a standing table adjustable in height and inclination, a fastening belt with holding devices on the standing table for the patient, a drive mechanism for the leg movement of the patient, consisting of a knee mechanism and a foot mechanism, wherein the standing table has a head portion displaceable with respect to a leg portion about a pivot joint, whereby the pivot joint provides an adjustable hip extension angle for which an adjusting mechanism is provided; and the knee portion and foot portion are displaceably arranged on rails on the leg mechanism; the foot mechanism serves to establish force on the sole of the foot during knee extension; a control unit is provided for controlling movement of the apparatus.

In yet another embodiment the automated movement therapy is carried out by employing a device for applying a force between first and second portions of an animate body, said device comprising: first and second link assemblies associated with said first and second portions, respectively, each said first and second link assembly comprising:
A. a supporting section secured in position on a portion, each supporting section being a supporting link; and
B. an articulated link attached through a joint to each of said supporting links; wherein said articulated links of said first and second link assemblies are attached to each other through a pivot joint, with said articulated link of said second assembly extending beyond said pivot joint; first and second casings attached to a link in said first link assembly; first and second tendons extending through said first and second casings, respectively, and attached to a link in said second link assembly, wherein one of said tendons is attached to said articulated link in said second link assembly on one side of said pivot joint and the other tendon is attached to said articulated link in said second link assembly on the opposite side of said pivot joint.

In another embodiment the automated movement therapy is carried out by employing a device for applying a force between first and second portions of a hand, one of said portions being a phalanx, said device comprising: first and second link assemblies associated with said first and second portions, respectively, each link assembly comprising:

a supporting section secured in position on a portion, each supporting section being a supporting link; and

an articulated link attached through a joint to each of said supporting links; wherein said articulated links of said first and second link assemblies are attached to each other through a pivot joint, with said articulated link of said second assembly extending beyond said pivot joint; first and second casings attached to a link in said first link assembly; and first and second tendons extending through said first and second casings, respectively, and attached to a link in said second link assembly, wherein one of said tendons is attached to said articulated link in said second link assembly on one side of said pivot joint and the other tendon is attached to said articulated link in said second assembly on the opposite side of said pivot joint.

In another embodiment the movement disorder is a hyperkinetic and/or hypokinetic movement disorder, wherein an imbalance between agonist and antagonist is interfering with function. In another embodiment the movement disorder is associated with cerebral palsy, M. Parkinson, central gait impairment, spinal cord injuries, dystonias, traumatic brain injury, genetic disorders, metabolic disorders, dynamic muscle contractures and/or stroke. In another embodiment the movement disorder is associated with at least one selected among pes equinus, pes varus, lower limb spasticity, upper limb spasticity, adductor spasticity, arm dystonia, hand dystonia, hip flexion contracture, hip adduction, knee flexion spasticity (crouch gait), plantar flexion of the ankle, supination and pronation of the subtalar joint, writer's cramp, musician's cramp, golfer's cramp, leg dystonia, thigh adduction, thigh abduction, knee flexion, knee extension, equinovarus deformity, foot dystonia, striatal toe, toe flexion, toe extension.

In one embodiment said patient is human. In another embodiment the patient has not completed its motor development and fixed muscle contractures have not occurred. In another embodiment the patient is a child up to six years in age.

In another embodiment the chemodenervating agent, e.g. botulinum toxin, is administered by injection.

In another embodiment said agent is administered several times during the treatment.

In another embodiment said agent is administered for the first time before commencement of the movement therapy.

In another embodiment said agent is re-administered in intervals of between 3 and 6 months.

In another embodiment said agent is re-administered in intervals of between 2 weeks and less than 3 months.

In another embodiment said agent is re-administered at a point in time when muscular activity interferes with the automated muscle activation therapy.

In one embodiment the chemodenervating agent is a botulinum toxin.

In one embodiment the effective amount of botulinum toxin administered exceeds 500 U of neurotoxic component in adults or exceeds 15 U/kg body weight in children.

In another embodiment the botulinum toxin is the botulinum toxin complex type A.

In another embodiment the botulinum toxin is the neurotoxic component of a Clostridium botulinum toxin complex.

In another embodiment the botulinum toxin is selected from the group consisting of serotypes A, B, C, D, E, F, G and a mixture thereof. In another embodiment the neurotoxic component is of type A.

Furthermore, the present invention relates to a kit for the treatment of patients suffering from movement disorders comprising a medicament comprising an effective amount of a chemodenervating agent and means for carrying out muscle stimulation therapy, such as devices for carrying out automated movement therapy.

In one embodiment said means for carrying out the muscle stimulation therapy is selected from the group of temperature stimulation means, electric stimulation means, vibration means, activation by sound-wave means, hydrostatic means, electro-magnetic wave means and magnetic field means, or any combination thereof.

In another embodiment said means for carrying out the muscle stimulation therapy is an automated movement therapy comprising a driven and controlled gait orthosis and/or arm mover which guides the extremities of said patient in a physiological pattern of movement.

In another embodiment the chemodenervating agent is a botulinum toxin.

In another embodiment the botulinum toxin is the neurotoxic component of a Clostridium botulinum toxin complex.

### DETAILED DESCRIPTION OF THE INVENTION

The present invention relates to the use of an effective amount of a chemodenervating agent for the manufacture of a medicament for administering to a patient for treating a movement disorder in said patient, wherein said patient is subjected to an, optionally automated, muscle stimulation therapy, and wherein said medicament is administered prior to and/or during and/or after said muscle stimulation therapy.

The term “(automated) muscle stimulation therapy” is herein under defined as any (automated) method able to provoke a muscle, either by automated muscle movement or by other means of automated muscle activation.

### A. Patient Group

The patient to be treated by the present method and kit can be of animal or human nature. In one embodiment, the patient is human. In another embodiment, the treatment is for a young patient, especially in regard to movement disorders associated with cerebral palsy. In this respect, the term “young” refers to a patient that has not completed its motor development and fixed muscle contractures have not occurred. In another embodiment, the patient can be a child of up to 6-8 years in age with unfinished motor development and motor maturation. In yet another embodiment, the children to be treated are up to six years old.

In another embodiment, the treatment of movement disorders involves symptoms of the underlying condition, e.g. CP, in particular symptoms which include difficulties with fine motor tasks (such as writing or using scissors), difficulty maintaining balance or involuntary movements. The symptoms may differ from person to person and may change over time.

In still another embodiment, the movement disorder is a hyperkinetic and/or hypokinetic movement disorder, wherein an imbalance between agonist and antagonist is interfering with muscle function.

In one embodiment of the present invention, the movement disorder involves spasticity of a muscle. In another embodiment of the present invention, the spasticity is, or is associated with, post-stroke spasticity or a spasticity caused by cerebral palsy.

“Spasticity” is defined as a motor disorder characterized by a velocity-dependent increase in tonic stretch reflexes (muscle tone) with exaggerated tendon jerks, resulting from hyperexcitability of the stretch reflex, as one component of the upper motor neuron syndrome. In some patients spasticity can be beneficial, as in the case of hip and knee extensor spasticity, which may allow weight bearing, with the affected limb acting like a splint. However, in the majority of patients spasticity causes difficulties with activities of daily living, such as dressing and cleaning the palm of the clenched hand. Some of the spasticity conditions that may involve movement disorders to be treated according to the present invention are exemplarily given in Table 1 below:

TABLE 1
Spasticity Conditions (selection)

| | | | | | |
|---|---|---|---|---|---|
| [symbol] | in encephalitis and | ▪ | autoimmune | ∘ | multiple sclerosis |
| | myelitis | | processes | ∘ | transverse |
| | | | | | myelitis |
| | | | | ∘ | devic syndrome |
| | | ▪ | viral infections | | |
| | | ▪ | bacterial infections | | |
| | | ▪ | parasitic infections | | |
| | | ▪ | fungal infections | | |
| [symbol] | in hereditary | | | | |
| | spastic paraparesis | | | | |
| [symbol] | in central nervous | ▪ | intracerebral | | |
| | system hemorrhage | | hemorrhage | | |
| | | ▪ | subarachnoidal | | |
| | | | hemorrhage | | |
| | | ▪ | subdural | | |
| | | | hemorrhage | | |
| | | ▪ | intraspinal | | |
| | | | hemorrhage | | |
| [symbol] | in neoplasias | ▪ | hemispheric tumors | | |
| | | ▪ | brainstem tumors | | |
| | | ▪ | myelon tumors | | |

The term “post-stroke spasticity” relates to spasticity occurring after a stroke incident. Stroke is a leading cause of long-term disability, with spasticity occurring in 19% to 38% of patients (Watkins C L, Leathley M J, Gregson J M, Moore A P, Smith T L, Sharma A K, Prevalence of spasticity post stroke, Clin Rehabil 2002; 16(5): 515-522. (ID 1915001)).

Cerebral palsy is an umbrella-like term used to describe a group of chronic disorders impairing control of movement that appear in the first years of life and generally do not worsen over time. The disorders are caused by faulty development or damage to motor areas in the brain that disrupts the brain's ability to control movement and posture.

It is our understanding that cerebral palsy cannot be treated, i.e. that the injury to the brain cannot be undone. Instead, it is intended to treat some symptoms of cerebral palsy, in particular those related to movement disorders. These symptoms are caused by an abnormal or disturbed muscle activity which prevents the affected muscles from relaxing. “Cerebral palsy” describes a wide spectrum of pyramidal dysfunctions causing paresis, extrapyramidal dysfunctions causing dystonia, rigidity, spasticity and spasms, apraxic components and coordinative dysfunctions. Cerebral palsy (Koman L A, Mooney J F, Smith B P, Goodman A, Mulvaney T. Management of spasticity in cerebral palsy with botulinum—A toxin: report of a preliminary, randomized, double-blind trial, J Pediatr Orthop 1994; 14(3): 299-303. (ID 1767458); Pidcock F S, The emerging role of therapeutic botulinum toxin in the treatment of cerebral palsy, J Pediatry 2004; 145(2 Suppl): S33-S35. (ID 2994781)) may occur after brain hemorrhage, asphyxia, premature birth or other perinatal complications. It is a life-long condition causing uncoordinated movements, paresis and various forms of muscle hyperactivity. Patients affected by cerebral palsy, when treated in accordance with the methods disclosed herein, experience a functional improvement of hyperactive muscles. It is, however, well within the scope of the present invention to improve muscle activity of muscles not affected by spasticity. This also includes coordination between different muscle groups. The term “muscle groups” includes adjacent muscles but also muscles in different body regions.

In accordance with the teaching of the present invention, common clinical patterns of movement disorders associated with spasticity in the corresponding muscle groups or movement disorders associated with cerebral palsy are treated by the method according to the invention, i.e. a combination of locomotion/movement therapy and administration of botulinum toxin.

In particular, the movement disorder is associated with cerebral palsy, M. Parkinson, central gait impairment, spinal cord injuries, dystonias, traumatic brain injury, genetic disorders, metabolic disorders, dynamic muscle contractures and/or stroke are treated, e.g. movement disorders which are associated with at least one selected among pes equinus, pes varus, lower limb spasticity, upper limb spasticity, adductor spasticity, hip flexion contracture, hip adduction, knee flexion spasticity (crouch gait), plantar flexion of the ankle, supination and pronation of the subtalar joint, writer's cramp, musician's cramp, golfer's cramp, leg dystonia, thigh adduction, thigh abduction, knee flexion, knee extention, equinovarus deformity, foot dystonia, striatal toe, toe flexion, toe extension.

### Muscle Activation Therapy

In said embodiment said any means being capable to stimulate, e.g. activate the target muscle may be used.

The term “muscle activation” thereby relates to any treatment of the muscle or muscle-group which increases the metabolism of this muscle or muscle-group above the average metabolic level of the same muscle if it is resting. For assessing the metabolism of a muscle the skilled artisan can for example measure the ATP production in the muscle, the activity of the creatine kinase, the glucose conversion and/or the fat conversion. Also indirect methods can be applied, e.g. the rise in muscle temperature, the rise of blood flow or measurement of muscle volume. However, it depends on the form of muscle-activation, the accessibility of the muscle and of the condition to be treated, which activity test(s) is(are) applied by the artisan. The term “muscle activation” also encompasses the activation of the moto-neuron, i.e. the elevation of the ability of the pre-synapse to take-up the chemodenervating agent. This activation might for example be noticeable by an elevated number of surface proteins e.g. receptors to which the chemodenervating agent is able to bind. Examples of such surface proteins are, for example, the SV2 protein (in all isoforms such as A, B, or C), polysialogangliosides (e.g. GT1b, GD1b, GQ1b) or synaptotagmin polypeptides (e.g. Syt1 or Syt2). It is clear to the artisan that different chemodenervating agents bind to different surface proteins. For example Botulinum toxin A is thought to bind to all SV2-isoforms, whereas Botulinum toxin B and G are thought to bind to Syt1 and Syt2. Elevated expression of surface proteins could for example be measured by biopsy and subsequent antibody staining against said proteins, or by the evaluation of levels of mRNA encoding for said proteins.

The term “automated muscle activation” relates to the process of activation the muscle with a technical device. In general, this muscle activation does not require active muscle movement of the patient, but the less active muscle is activated by said technical device.

The term “means for muscle activation” relates to any means suitable to activate the muscle, as they are disclosed herein under. “Means” therefore comprises a technical device, an agent or a physical stimulation of the muscle e.g. via massage, temperature, electrical stimulation, electro-magnetic waves, sound waves, vibration, etc.

Without being restricted any of the following methods, some methods to stimulate muscles are summarized herein under.

### B.1 Activation by Temperature Stimulation

Changed temperature conditions can be used to activate the muscle in the desired way. It is known that low temperatures lead to micro-contractions of the muscles in order to keep the body temperature in a certain range. On the other hand elevated temperatures lead to vascular dilatation and better supply of the muscles with oxygen and nutrients, therefore also leading to a more active muscle. Generally, muscle temperature may be adjusted by any means capable of heating and/or cooling muscles.

In one embodiment heat is applied to the muscle via infra-red light. Normally light bulbs, which emit IR-A-light (wavelength between 700 nm-1400 nm), are used to warm up the local tissue and enhance blood-flow and relaxation, therefore stimulating and activating the muscle.

In another embodiment the cooling or heating of the muscle is facilitated by the use of a water bath. In another embodiment an air-stream is used to heat or cool the target muscle.

In another embodiment the heating and cooling can be enhanced by the administration of a chemical to the skin surface, thereby allowing for a faster and a more focused cooling or heating of the target muscle. Feasible chemicals for cooling are for example those which create enhanced evaporation on the skin (e.g. chloroethylspray, alcohol based ice-sprays or cooling gels). Feasible chemicals for heating are for example such, which created enhanced blood flow and/or heat sensation on the skin (e.g. Capsicain, Nonivamid etc.).

In another embodiment the heating and/or cooling of the muscle is facilitated by compresses or “heating-” or “cooling-packs”, i.e. materials which are either heated up or cooled down externally and are able to keep a stable temperature over a certain time period (e.g. fango-packs, temperature) or the heat or coldness is produced by a chemical or physical reaction (e.g. an endothermal or exothermal reaction).

In another embodiment, the temperature-levels are shifted periodically between cold and warm temperatures. In general “heating” is herein under defined as elevating the muscle temperature above 40° C., above 45° C. above 50° C., above 55° C., up to 60° C., up to 70° C. or up to 80° C. On the other hand “cooling” of a muscle is defined herein under as lowering the temperature below 35° C., below 30° C., below 25° C., below 20° C., below 10° C., down to 0° C., down to −10° C. or down to below −20° C. It depends on the individual sensitivity of the patient, the size of the muscle and the time period of application to decide, which heat and coldness is still feasible to apply.

### B.2 Activation by Electric Stimulation

Within the present invention any method/means capable of electrically stimulating muscles may be used. Some examples are discussed herein under in more detail:

In one embodiment the muscle activation is facilitated by functional electrical stimulation (commonly abbreviated as FES). This is a technique that uses electrical currents to activate nerves innervating extremities affected by paralysis resulting from spinal cord injury (SCI), head injury, stroke or other neurological disorders, restoring function in people with disabilities.

Normally two ways of functional electrical stimulation can be applied:

In one embodiment the nerves are stimulated. In this case the electric field strength has to be applied with a gradient strong enough to elicit an action potential within the targeted motoric nerve.

In another embodiment the muscles are stimulated directly. In this case stronger and longer stimulation impulses are needed in comparison with the stimulation of nerves to elicit an activation of the muscle.

In both cases stimulation can be applied via surface-electrodes or implanted electrodes (e.g. in cases of chronical spasticity). A suitable cream should be used to increase conductivity from the electrode to the skin. The position of the electrodes on the skin determines which nerve(s), respectively muscle(s) is (are) stimulated.

In another embodiment the electrical stimulation is facilitated by a transcutaneous electrical nerve stimulator, more commonly referred to as a TENS. This is an electronic device that produces electrical signals used to stimulate nerves through unbroken skin. The unit may be connected to the skin using two or more electrodes. A typical battery-operated TENS unit consists of a pulse generator, small transformer, frequency and intensity controls, and a number of electrodes. The electrodes are attached to an implanted receiver, which receives its power from an antenna worn on the surface of the skin. This application of an implanted device might be useful for the treatment of patients with chronic spasticities.

In another embodiment the electrical stimulation is facilitated by high-frequent muscle stimulation. Alternating electrical fields of high frequency of 4 to approximately 30 kHz (kilo-Hz) may be used. Furthermore, both the intensity as well as the frequency of the electric current may be modulated. This leads to activating effects on the nerves and adjacent muscles.

In another embodiment the electrical stimulation is facilitated by the interference-therapy (also called NEMEC-therapy). In this case electrical currents of intermediate frequency are used, which interfere inside the target tissue and are thought to elicit endogenous stimulations of muscles and nerves.

### B.3 Activation by Vibration

According to the present invention muscles may be stimulated by whole-body or local stimulation, i.e. stimulation of individual muscles or muscle groups.

In one embodiment the muscle activation is facilitated by technical devices, which introduce vibrations into the body.

In one embodiment the whole-body vibration (WBV) is used. WBV is the human exposure to vibration through feet, buttock and/or back. In WBV, the entire body is exposed to vibration, as opposed to local vibration (biomechanical stimulation, BMS), where an isolated muscle or muscle group is stimulated by the use of a vibration device. The vibrations the engines generate are transmitted to the person standing, sitting or lying on the machine. The intensity and the direction of these vibrations are essential for their effect. The skilled artisan will understand, that he has to adapt the vibration intensity and direction according to the muscle to be treated.

In order to elicit a stretch reflex in the muscles the up-down movement is the most important. Human bodies are made to absorb better vertical vibrations due the gravity effect. However, many machines vibrate in three different directions: sideways (x), front and back (y) and up and down (z), which could cause significant side effects after prolonged time of use. The z-axis has the largest amplitude and is the most defining component in generating and inducing muscle contractions. Concerning the z-movements two principle types of systems can be distinguished: side alternating systems, operating like a see-saw and hence mimicking the human gait where always one foot is moving upwards and the other one down-wards and systems where the whole platform is mainly doing the same motion respectively both feet are moved upwards or downwards at the same time. Systems with side alternation offer a larger amplitude of oscillation and a frequency range of about 5 Hz to 35 Hz the other systems offer lower amplitudes but higher frequencies in the range of 20 Hz to 50 Hz. Despite the larger Amplitudes of side-alternating systems the vibration (acceleration) transmitted to the head is significantly lower then in non side-alternating systems.

The mechanical stimulation generates acceleration forces working on the body. it is believed that these forces cause the muscles to lengthen, and this signal is received by the muscle spindle, a small organ in the muscle. This spindle transmits the signal through the central nervous system to the muscles involved. Due to this subconscious contraction of the muscles, many more muscle fibers are used than in a conscious, voluntary movement.

In one another embodiment the vibration device is for example the Hand-Arm Vibration (HAV), where the vibration is transferred through a limb, i.e. the hand and arm or foot and leg. In another embodiment very small muscles, e.g. in the face, are activated via local vibration devices which stimulate the muscle in a small area of a few centimeters in diameter.

Typically, muscle stimulation takes place between 8 and 45 Hz.

Three groups of vibration can be exemplified:

Below 10-12 Hz the postular stability control system is activated, therefore muscles of the postular system are activated.

Between 12 Hz and approximately 20 Hz the reflex-based system (a feedback via the muscle spindle and the spinal cord) is activated. This allows for a cycle of contraction and relaxation in the muscle.

Above 20 Hz the time for relaxation of the muscle is too short, therefore the muscle contracture will increase during the treatment. An increase of frequency above 40 Hz seems only in few cases appropriate, normal ranges are below 20 to 30 Hz, 20 to 35 Hz.

### B.4 Activation by Sound-Waves

In one embodiment the muscle activation is facilitated by sound waves.

In one embodiment said sound waves are therapeutic ultrasound waves (range of 20 kHz to 10 GHz).

In one embodiment the frequency of the used therapeutic ultrasound is between 1 to 3 MHz. At this frequency, the waves tend to travel through tissue with high water or low protein content, they are reflected by cartilage and bone. They are absorbed primarily by connective tissue: ligaments, tendons, and fascia (and also by scar tissue). In this embodiment the therapeutic ultrasound seems to have two types of benefit.

Thermal effects involve energy absorbed from the sound waves heating the target muscle, which leads to its activation.

Cavitational effects result from the vibration of the tissue causing microscopic air bubbles to form, which transmit the vibrations in a way that directly stimulates the cell membranes of the muscle, which also leads to an activation of the muscle.

In another embodiment HIFU (high intensity focused ultrasound) (sometimes FUS or HIFUS) is used. In HIFU a high-intensity focused ultrasound is used to heat tissue rapidly. Although it is normally used to destroy pathogenic tissue, it can also be used with lower intensities to rapidly heat a muscle (without destroying it), thereby activating said muscle. If necessary this activation can be guided by computerized MRI. In these cases it is referred to as Magnetic Resonance guided Focused Ultrasound, often shortened to MRgFUS.

In another embodiment, the applied sound is acoustical sound, e.g. has a frequency between 20 Hz and 20000 Hz. At these frequencies, the vibrational effects of the sound are used to induce muscle movements.

In one embodiment standing waves, also known as a stationary waves, are used to e.g. induce cellular movements of the muscle fibers. This phenomenon seems to occur because the medium (muscle, tissue-liquids, etc.) is moving in the opposite direction to the wave. The activation is also a result of interference between two waves traveling in opposite directions. The intensity and frequency of the sound has to be adjusted according to the length and size of the target muscle.

In another embodiment an ultrasound-device is used to not only activate the muscle, but also assist in guiding the injection needle containing the chemodenervating agent to the site of application. In this embodiment the application of the chemodenervating agent may be divided in three steps:
A. Identification of the muscle spasm via normal ultrasound imaging technology.
B. Application of a focused ultrasound to activate the muscle at the site of the spasm.
C. Guidance of the needle containing the chemodenervating agent for injection into the identified site being for example a muscle with or without a spasm or fibrosis via normal ultrasound imaging technology.

### B.5 Activation by Hydrostatic Means

In one embodiment the muscle activation is facilitated by hydrostatic means. In one embodiment said hydrostatic means are water-jets which are used for muscle activation. In another embodiment said mechanical means is a subaqueous-pressure-stream-massage (Unterwasserdruckstrahlmassage, UWM). In this embodiment a special bathtub is used which is connected to a pump which circulates the water of the bathtub through a water hose. Therefore the jet temperature is the same like the water temperature of the bathtub. However, via the pump-unit additional water can be added to the water stream to change the temperature of the massage jet. The pump normally applies a pressure of 0.5 to 3 bar, which allows, depending on the diameter of the used water hose and type of nozzle, for different massage techniques. The regulation of the intensity of the jet and/or diameter of hose and nozzle allow for an adjustment to the target muscle.

### B.6 Activation by Electro-Magnetic Waves or Magnetic Fields

In one embodiment the muscle activation is facilitated by electro-magnetic waves.

In one embodiment microwaves of low intensity are used to induce heat in a muscle and therefore activate the muscle. Microwaves are electromagnetic waves with wavelengths shorter than one meter and longer than one millimeter, or frequencies between 300 megahertz and 3 gigahertz.

In another embodiment the muscle activation is facilitated via repetitive magnetic muscle stimulation (cf. e.g. Swallow et al., J. Appl. Physiol. 2007; 103: 739-746). In this embodiment a large, flexible oval coil is used, which could be wrapped securely over the front of the thigh. The coil is fluid cooled to prevent overheating. In magnetic stimulation, a quickly changing magnetic field is generated by a pulse of current flowing through a coil of wire. The magnetic field in turn generates a current inside the body, and this depolarizes axons in the same way as an electrical stimulus. The advantage is that with magnetic stimulation the current does not have to pass through the relative high resistance of the skin so that nociceptors in the skin are not activated. For stimulating a quadriceps muscle for example 30 Hz with an intensity sufficient to produce 30% of the maximal twitch force and a pattern of 2 seconds contraction and 3 seconds rest were applied.

In another embodiment transcranial magnetic stimulation (TMS) is used to activate the target muscle. TMS is a noninvasive method to excite neurons in the brain: weak electric currents are induced in the tissue by rapidly changing magnetic fields (electromagnetic induction). This way, for example in the motoric centers of the brain, brain activity can be triggered with minimal discomfort, and the associated muscle will be activated. In another embodiment repetitive transcranial magnetic stimulation, known as rTMS, can produce longer lasting activation of muscles and therefore seems to be more feasible for certain patient conditions.

### B.7 Pharmaceutical Muscle Activation Therapy

In another embodiment the above recited muscle activation may also be achieved by applying pharmaceuticals to the muscle. Said “muscle activating pharmaceuticals” are substances able to set the muscle into a state of “muscle activity” as defined under C above. The term “pharmaceutical” herein under is defined as any substance which is able to change the condition of said muscle into an activated state.

In one embodiment the muscle activating pharmaceutical is a stimulant. The term “stimulant” is defined as any substance, especially a chemical agent that temporarily arouses or accelerates physiological or organic activity, e.g. a β₃ agonist, caffeine, ephedrine (e.g. Ma Huang), amphetamine, methamphetamine, methylphenidate, and/or cocaine-derivate

In another embodiment the muscle activating pharmaceutical is a muscle contractant. The term “muscle contractant” is defined herein under as any substance able to induce contraction in a muscle e.g. any substance with sympathetic effect, any substance with agonistic effects on 2-adrenergic receptor, caffeine, acetylcholine, nicotine, epibatidine-derivatives (e.g. ABT-594), dimethylphenylpiperazinium, succinyl choline, a muscle stimulating saponin-derivative [(e.g. isolated from Dalbergia saxatilis), cf. C. N. Uchendu^{a,}*- and B. F. Leek^{b} Fitoterapia Volume 70, Issue 1, 1 Feb. 1999, Pages 50-53], calcium, potassium, norepinephrine, adrenaline (epinephrine), leukotrienes, allene containing arachidonic acid

In a further embodiment the muscle activating pharmaceutical is a substance which increases blood flow within the muscle, e.g. EDHF, interstitial K⁺, nitric oxide, β² adrenergic agonists, histamine, prostacyclin, prostaglandin, VIP, (extracellular) adenosine, (extracellular) ATP, (extracellular) ADP, L-Arginine, bradykinin, substance P, niacin (nicotinic acid), platelet activating factor (PAF), CO₂, interstitial lactic acid, Adenocard®, alpha blockers, amyl nitrite, atrial natriuretic peptide, ethanol, histamine-inducers (e.g. complement proteins C3a, C4a and C5a), nitric oxide inducers (e.g. glyceryl trinitrate (commonly known as nitroglycerin), isosorbide mononitrate, isosorbide dinitrate, pentaerythritol tetranitrate (PETN), sodium nitroprusside, PDE5 inhibitors, agents which indirectly increase the effects of nitric oxide (e.g. sildenafil (Viagra®), tadalafil, tardenafil), tetrahydrocannabinol, theobromine and/or papaverine.

In another embodiment the muscle activating pharmaceutical is a substance which increases the muscle temperature directly or indirectly (i.e. via increasing the core body temperature), i.e. act thermogenic in the patient. Examples for such substances are ephedra, bitter orange (synephrine), capsicum, ginger, sibutramine and its metabolites and/or caffeine.

In another embodiment the muscle activating pharmaceutical is a substance which up-regulates the number of surface proteins (e.g. receptors) thereby allowing the chemodenervating agent to bind and enter the cell, typically the presynaptic cell. In one embodiment said substance up-regulates SV2 of the muscle enervating neuron at the presynapsis. In another embodiment polysialogangliosides (e.g. GT1b, GD1b, GQ1b) or synaptotagmin polypeptides (e.g. Syt1 or Syt2) are upregulated. Examples for such substances are hormones, growth factors, neurotrophins, blocking substances of receptor-internalization, factors which enhance the receptor surface expression, arrestin-inhibitors, protease inhibitors, blocking substances of receptor degradation, inhibitors of inhibitory G-proteins, competitive receptor antagonists, and/or neurotransmitter degrading agents.

### B. 8 Automated Movement Therapy

In one embodiment one part of the treatment is the so-called automated movement therapy. The term “movement therapy” relates to any kind of therapy, wherein the patient is trained to move its extremities in a “normal” manner, i.e. in a physiological movement or a sequence of movements. The therapy of upper and lower extremities shall be included in the term “movement therapy”. The term “automated movement therapy” relates to any kind of movement therapy wherein the patient is trained to use its muscles in a “normal” manner, i.e. a physiological (sequence of) movement(s), by way of an automated (driven) orthotic device. The movement therapy being part of the present invention encompasses, but is not limited to, the selective movement of upper and lower extremities and/or parts thereof, such as arms and legs as well as the shoulder, elbow, wrist, hand, finger, thumb, knee, feet, and toe joints in multiple ways, either isolated or within movement chains.

In general, a device which is to be used within the method and the kit of the present invention comprises a driven and controlled orthotic device, which guides the extremities, e.g. the legs or arms of the patient, in a physiological pattern of movement. Preferably, the device is supported by an automated gait orthosis or an arm mover.

For patients having problems with their leg movement, the device preferably comprises an automated gait orthosis, more preferably in combination with a treadmill. Respective devices for an automated movement (locomotion) therapy are meanwhile commercially available, for example from the company Hocoma AG under the trademark Lokomat®. Such devices, based on the treadmill therapy are described in detail in U.S. Pat. No. 6,821,233 and the prior art described therein, which is fully incorporated by reference herein.

Thus, said automated movement therapy can be carried out by using a device comprising a driven and controlled orthotetic device which guides the legs of said patient in a physiological pattern of movement, a treadmill and, preferably, a relief mechanism acting on the body weight of said patient. Preferably, such a device is used for treating gait movement disorders.

In particular, within the method of the present invention, use is made of the devices as described in U.S. Pat. No. 6,821,233. One device is an apparatus for treadmill training, comprising a treadmill, a relief mechanism for the patient, and a driven orthotic device, wherein a parallelogram fixed in a height-adjustable manner on the treadmill is provided for stabilizing the orthotic device and preventing the patient from tipping forward, backwards and sidewards, the parallelogram being attached to the orthotic device. The orthotic device preferably comprises a hip orthotic device and two leg parts, whereby two hip drives are provided for moving the hip orthotic device, and two knee drives are provided for moving the leg parts; the hip orthotic device and leg parts are adjustable, the leg parts are provided with cuffs which are adjustable in size and position; and a control unit is provided for controlling the movements of the orthotic device and controlling the speed of the treadmill.

Alternatively, another device is an apparatus for treadmill training, comprising a treadmill including a railing, a relief mechanism for the patient, and a driven orthotic device, wherein means for stabilizing the orthotic device are provided that prevent the patient from tipping forward, backwards and sidewards. The orthotic device preferably comprises a hip orthotic device and two leg parts, two hip drives are provided for moving the hip orthotic device, and two knee drives are provided for moving the leg parts; a ball screw spindle drive is provided for each knee drive and hip drive, the orthotic device and leg parts are adjustable, the leg parts are provided with cuffs which are adjustable in size and position; a control unit is provided for controlling the movements of the orthotic device and controlling the speed of the treadmill can be used. Both devices are described in detail in U.S. Pat. No. 6,821,233.

In these devices, the relief force is provided by attaching a roller above the base frame, over which roller a wire cable is passed that is attached near the bearing and is loaded on the other side of the parallelogram with a counterweight (see FIG. 1 of U.S. Pat. No. 6,821,233). An improved device for adjusting the height of and the relief force acting on the weight of a patient is the subject of EP 1 586 291 A1, which is also incorporated herein by reference. The device for adjusting the height of and the relief force acting on the weight of the patient is characterized in that said weight is supported by a cable, with a first cable length adjustment means to provide an adjustment of the length of the cable to define the height of said suspended weight and a second cable length adjustment means to provide an adjustment of the length of the cable to define the relief force acting on the suspended weight.

In a further embodiment of the present invention, the movement therapy is carried out by using an apparatus for locomotion therapy for the rehabilitation of paraparetic and hemiparetic patients, comprising a standing table that is preferably adjustable in height and inclination, a fastening belt with holding devices on the standing table for the patient, a drive mechanism for the leg movement of the patient, consisting of a knee mechanism and a foot mechanism, wherein the standing table has a head portion displaceable with respect to a leg portion about a pivot joint, whereby the pivot joint provides an adjustable hip extension angle for which an adjustable mechanism is provided. Preferably, the knee portion and foot portion are displaceably arranged on rails on the leg mechanism. Preferably, the foot mechanism serves to establish force on the sole of the foot during knee extension. Preferably, a control unit is provided for controlling the movement of the apparatus. Further details regarding said apparatus and its method of operation can be derived from U.S. Pat. No. 6,685,658, which is again incorporated herein by reference in full.

In another embodiment of the present invention, in regard to the movement therapy, use is made of a device for applying a force between first and second portions of an animate body, said device comprising: first and second link assemblies associated with said first and second portions, respectively, each of said first and second link assembly preferably comprising: (a) a supporting section secured in position on a portion, each supporting section being a supporting link; and (b) an articulated link attached through a joint to each of said supporting links; wherein said articulated links of said first and second link assemblies are attached to each other through a pivot joint, with said articulated link of said second assembly extending beyond said pivot joint; first and second casings attached to a link in said first link assembly; first and second tendons extending through said first and second casings, respectively, and attached to a link in said second link assembly, wherein one of said tendons is attached to said articulated link in said second link assembly on one side of said pivot joint and the other tendon is attached to said articulated link in said second link assembly on the opposite side of said pivot joint.

Alternatively, use is made of a device for applying a force between first and second portions of a hand, one of said portions being a phalanx, said device comprising: first and second link assemblies associated with said first and second portions, respectively; each link assembly preferably comprising: (a) a supporting section secured in position on a portion, each supporting section being a supporting link; and (b) an articulated link attached through a joint to each of said supporting links; wherein said articulated links of said first and second link assemblies are attached to each other through a pivot joint, with said articulated link of said second assembly extending beyond said pivot joint; first and second casings attached to a link in said first link assembly; and first and second tendons extending through said first and second casings, respectively, and attached to a link in said second link assembly, wherein one of said tendons is attached to said articulated link in said second link assembly on one side of said pivot joint and the other tendon is attached to said articulated link in said second assembly on the opposite side of said pivot joint. Further details as to the device as such and the method of its use may be derived from U.S. Pat. No. 6,059,506, which is again incorporated herein by reference in full.

Within the scope of the present invention, any other commercially available device or any device as developed in academic facilities and suited for automated locomotion therapies may be used. In this respect, mention is made of the “MIT-Manus” device, the “Mirror-Image Motion Enabler” (MIME) robot, the “ARM guide”, the “Bi-Manu-Track” arm trainer, the GTI, an electromechanical gait trainer, and the NeRobot and REHAROB devices. More details regarding these devices may be taken from Hesse S, Schmidt H, Werner C, Bardeleben A. (2003), Upper and lower extremity robotic devices for rehabilitation and for studying motor control, Curr Opin Neurol 16: 705-710) and the literature cited therein.

More preferably, devices as commercially available from the company Hocoma AG are used, in particular those commercialized under the trademark Lokomat® and Armeo® at the filing date of the present application.

### B.9 Combination of Muscle Activation

It is clear to the artisan that the above mentioned means of muscle activation can be combined according to the special needs of the patient. For example hydrostatic activation can be combined with temperature activation of the muscle, electrical activation can be combined with pharmaceutical activation, magnetic activation can be combined muscle movement activation, etc.

Also the means of muscle activation can be applied in varying time-intervals, e.g. within 1 to 100 milliseconds, 100 milliseconds to 1 second, 1 second to 5 seconds, 5 seconds to 30 seconds, 30 seconds to 1 minute, 1 minute to 10 minutes, 10 minutes to 30 minutes, 30 minutes to 1 hour, 1 hour to 12 hours, 12 hours up to 1 day, up to 10 days, up to 1 month, up to one year. These time intervals are just for exemplary purpose, therefore any time interval in-between is also encompassed.

### Administration of the Chemodenervating Agent

As recited above, in accordance with the present invention, the muscle stimulation, e.g. the automated movement therapy or the automated muscle activation therapy, is used in combination with the administration of an effective amount of a chemodenervating agent, in one embodiment a botulinum toxin e.g. in a form of a pharmaceutical composition/medicament, to said patient, wherein the administration is carried out prior to and/or during and/or after the muscle stimulation, e.g. movement or activation, therapy.

### D.1 The Chemodenervating Agent

In one embodiment said chemodenervating agent is a Clostridium neurotoxin. In a further embodiment this Clostridium neurotoxin is a botulinum toxin. In an even further embodiment the botulinum toxin is botulinum toxin of the antigenically distinct serotypes A, B, C, D, E, F, or G. Wherever the botulinum toxin serotype A, B, C, D, E, F or G are mentioned, also known variants of the serotypes are encompassed, like serotypes A1, A2, A3, B1, B2, B3, C1, C2, C3, D1, D2, D3, E1, E2, E3, F1, F2, F3, or G1, G2, G3. In one embodiment the botulinum toxin is botulinum toxin A.

In another embodiment, also isoforms, homologs, orthologs and paralogs of botulinum toxin are encompassed, which show at least 50%, at least 60%, at least 70%, at least 80%, at least 90% or up to 100% sequence identity. The sequence identity can be calculated by any algorithm suitable to yield reliable results, for example by using the FASTA algorithm (W. R. Pearson & D. J. Lipman PNAS (1988) 85:2444-2448).

Botulinum toxins, when released from lysed Clostridium cultures are generally associated with other bacterial proteins, which together form of a toxin complex. In a further embodiment said botulinum toxin is free of any complexing proteins, e.g. it is the pure neurotoxin serotype A. In addition thereto, modified as well as recombinant produced neurotoxic components of botulinum toxins including the respective mutations, deletions, etc. are also within the scope of the present invention. With respect to suitable mutants, reference is made to WO 2006/027207 A1, WO 2006/114308 A1 and EP07014785.5 (patent application by Merz, filed on Jul. 27, 2007) which are fully incorporated by reference herein. Furthermore, within the present invention, mixtures of various serotypes (in the form the neurotoxic component or recombinant form or both forms thereof, e.g. mixtures of botulinum neurotoxins of types A and B) may be used. The present invention, however, also refers to neurotoxins which are chemically modified, e.g. by pegylation, glycosylation, sulfatation, phosphorylation or any other modification, in particular of one or more surface or solvent exposed amino acid(s).

The terms “botulinum toxin” or “botulinum toxins” as used throughout the present application, refer to the neurotoxic component devoid of any other clostridial proteins, but also to the “botulinum toxin complex”: The term “botulinum toxin” is used herein in cases when no discrimination between the complex or the neurotoxic component is necessary or desired. “BoNT” or “NT” are commonly used abbreviations. The complex usually contains additional, so-called “non-toxic” proteins, which are referred to herein as “complexing proteins” or “bacterial proteins”.

The complex of neurotoxic component and bacterial proteins is referred to as “Clostridium botulinum toxin complex” or “botulinum toxin complex”. The molecular weight of this complex may vary from about 300,000 to about 900,000 Da. The complexing proteins are, for example, various hemagglutinins. The proteins of this toxin complex are not toxic themselves but provide stability to the neurotoxic component during passage through the gastrointestinal tract. Medicaments on the basis of the botulinum toxin complex of types A, B and C are commercially available, type A botulinum toxin from Ipsen (under the trademark Dysport®) and from Allergan Inc. under the trademark Botox®.

The neurotoxic subunit of this complex is referred herein as the “neurotoxic component” of the botulinum toxin complex. The neurotoxic component of the botulinum toxin complex is initially formed as a single polypeptide chain, having, in the case of serotype A, a molecular weight of approximately 150 kDa. In other serotypes, the neurotoxic component has been observed to vary between about 145 and about 170 kDa, depending on the bacterial source.

In the case of serotype A, for example, proteolytic processing of the polypeptide results in an activated polypeptide in the form of a dichain polypeptide, consisting of a heavy chain and a light chain, which are linked by a disulfide bond. In humans, the heavy chain mediates binding to pre-synaptic cholinergic nerve terminals and internalization of the toxin into the cell. The light chain is believed to be responsible for the toxic effects, acting as zinc-endopeptidase and cleaving specific proteins responsible for membrane fusion (SNARE complex) (see e.g. Montecucco C., Shiavo G., Rosetto O: The mechanism of action of tetanus and botulinum neurotoxins, Arch Toxicol. 1996; 18 (Suppl.): 342-354).

By disrupting the process of membrane fusion within the cells, botulinum toxins prevent the release of acetylcholine into the synaptic cleft. The overall effect of botulinum toxin at the neuro-muscular junction is to interrupt neuro-muscular transmission, and, in effect, denervate muscles. Botulinum toxin also has activity at other peripheral cholinergic synapses, causing a reduction of salivation or sweating.

Each serotype of botulinum toxin binds to the serotype specific receptor sites on the pre-synaptic nerve terminal. The specificity of botulinum toxin for cholinergic neurons is based on the high affinity of the heavy chain for the receptor sites on these nerve terminals (Ref.: Katsekas S., Gremminloh G., Pich E. M.: Nerve terminal proteins; to fuse to learn. Transneuro Science 1994; 17: 368-379).

The term “neurotoxic component” also includes functional homologs found in the other serotypes of Clostridium botulinum. In one embodiment of the present invention, the neurotoxic component is devoid of any other C. botulinum protein, in one embodiment also devoid of RNA, which might potentially be associated with the neurotoxic component. The neurotoxic component may be the single chain precursor protein of approximately 150 kDa or the proteolytically processed neurotoxic component, comprising the light chain (L_{c}) of approximately 50 kDa and the heavy chain (H_{c}) of approximately 100 kDa, which may be linked by one or more disulfide bonds (for a review see e.g. Simpson L L, Ann Rev Pharmacol Toxicol. 2004; 44:167-93).

Within this invention, all forms of botulinum toxin, in particular the various serotypes, the various complexes of the neurotoxic component of botulinum toxin and its complexing accompanying proteins and the neurotoxic component of these botulinum toxins are to be used. In addition thereto, modified and/or recombinantly produced botulinum toxins or neurotoxic components of botulinum toxins including the respective mutations, deletions, etc. are also within the scope of the present invention. With respect to suitable mutants, reference is made to WO 2006/027207 A1, which is fully incorporated by reference herein. Furthermore, within the present invention, mixtures of various serotypes (in the form of the complex, the neurotoxic component and/or recombinant form), e.g. mixtures of botulinum toxins of types A and B or mixtures of botulinum neurotoxins of types A and B are also to be used.

In accordance with the teaching of the present invention it is possible that the medicament contains no proteins found in the botulinum toxin complex other than the neurotoxic component. The precursor of the neurotoxic component may be cleaved or uncleaved, however, in one embodiment the precursor has been cleaved into the heavy and the light chain. As pointed out elsewhere herein, the polypeptides may be of wild-type sequence or may be modified at one or more residues. Modification comprises chemical modification e.g. by glycosylation, acetylation, acylation or the like, which may be beneficial e.g. to the uptake or stability of the polypeptide. The polypeptide chain of the neurotoxic component may, however, alternatively or additionally be modified by addition, substitution or deletion of one or more amino acid residues.

### D.2 The Pharmaceutical Composition

### D.2.1 The Neurotoxic Component

The neurotoxic component referred to herein above, may be part of a composition or a pharmaceutical composition. This pharmaceutical composition to be used herein may comprise botulinum toxin, e.g. in the form of neurotoxic component as the sole active component or may contain additional pharmaceutically active components e.g. a hyaluronic acid or a polyvinylpyrrolidone or a polyethleneglycol, such composition being optionally pH stabilized by a suitable pH buffer, in particular by a sodium acetate buffer, and/or a cryoprotectant polyalcohol.

In one embodiment, the neurotoxic component has a biological activity of 50 to 250 LD₅₀ units per ng neurotoxic component, as determined in a mouse LD₅₀ assay. In another embodiment, the neurotoxic component has a biological activity of about 150 LD₅₀ units per nanogram. Generally, the pharmaceutical composition of the present invention comprises neurotoxic component in a quantity of about 6 pg to about 30 ng.

A “pharmaceutical composition” is a formulation in which an active ingredient for use as a medicament or a diagnostic is contained or comprised. Such pharmaceutical composition may be suitable for diagnostic or therapeutic administration (i.e. by intramuscular or subcutaneous injection) to a human patient.

A pharmaceutical composition comprising the neurotoxic component of botulinum toxin type A in isolated form is commercially available in Germany from Merz Pharmaceuticals GmbH under the trademark Xeomin®. The production of the neurotoxic component of botulinum toxin type A and B are described, for example, in the international patent applications WO 00/74703 and WO 2006/133818.

In one embodiment, said composition comprises the neurotoxic component of botulinum toxin type A. Said composition is a reconstituted solution of the neurotoxic component of botulinum toxin. In another embodiment the composition further comprises sucrose or human serum albumin or both, still another embodiment the ratio of human serum albumin to sucrose is about 1:5. In one embodiment, the composition is Xeomin®. In another embodiment, said human serum albumin is recombinant human serum albumin. Alternatively, said composition is free of mammalian derived proteins such as human serum albumin. Any such solution may provide sufficient neurotoxin stability by replacing serum albumin with other non-proteinaceous stabilizers (infra).

Within the present patent application, the use of a medicament based on the neurotoxic component of botulinum toxin type A, in another embodiment the product distributed by Merz Pharmaceutical under the trademark Xeomin® can be used. This is because the tendency of generating antibodies within the patient was found to be lower when applying pharmaceutical compositions on the basis of the neurotoxic component of botulinum toxin, such as Xeomin® compared to administering medicaments on the basis of the botulinum toxin type A complex. Without being bound to any theory, it is believed that the hemagglutinins within the botulinum toxin complex have an activating capability on the immune system.

With regard to the composition and dosing of the medicament on the basis of botulinum toxin, and in regard to the composition, dosing and frequency of administration of the medicament on the basis of the neurotoxic component of botulinum toxin, reference is made to PCT/EP2007/005754.

The pharmaceutical composition may be lyophilized or vacuum dried, reconstituted, or may prevail in solution. When reconstituted, in one embodiment the reconstituted solution is prepared adding sterile physiological saline (0.9% NaCl).

### D.2.2 Additional Components (“Excipients”)

Such composition may comprise additional excipients. The term “excipient” refers to a substance present in a pharmaceutical composition other than the active pharmaceutical ingredient present in the pharmaceutical composition. An excipient can be a buffer, carrier, antiadherent, analgesic, binder, disintegrant, filler, diluent, preservative, vehicle, cyclodextrin and/or bulking agent such as albumin, gelatin, collagen, sodium chloride, preservative, cryoprotectant and/or stabilizer.

D.2.3 pH-Buffers

A “pH buffer” refers to a chemical substance being capable to adjust the pH value of a composition, solution and the like to a certain value or to a certain pH range. In one embodiment this pH range can be between pH 5 to pH 8, in another embodiment pH 7 to pH 8, in yet another embodiment 7.2 to 7.6, and in yet a further embodiment a pH of 7.4. In another embodiment the pharmaceutical composition has a pH of between about 4 and 7.5 when reconstituted or upon injection, in yet another embodiment about pH 6.8 and pH 7.6 and in a further embodiment between pH 7.4 and pH 7.6.

In one embodiment the composition also contains a 1-100 mM, in another embodiment 10 mM sodium acetate buffer.

The pH ranges given mentioned above are only typical examples and the actual pH may include any interval between the numerical values given above. Suitable buffers which are in accordance with the teaching of the present invention are e.g. sodium-phosphate buffer, sodium-acetate buffer, TRIS buffer or any buffer, which is suitable to buffer within the above pH-ranges.

### D.2.4 Stabilizers

“Stabilizing”, “stabilizes” or “stabilization” means that the active ingredient, i.e., the neurotoxic component in a reconstituted or aqueous solution pharmaceutical composition has greater than about 20%, 30%, 40%, 50%, 60%, 70%, 80%, 90%, and up to about 100% of the toxicity that the biologically active neurotoxic component had prior to being incorporated into the pharmaceutical composition.

Examples of such stabilizers are gelatin or albumin, in one embodiment of human origin or obtained from a recombinant source. Proteins from non-human or non-animal sources are also included. The stabilizers may be modified by chemical means or by recombinant genetics. In one embodiment of the present invention, it is envisaged to use alcohols, e.g., inositol, mannitol, as cryoprotectant excipients to stabilize proteins during lyophilization.

In another embodiment of the present invention, the stabilizer may be a non proteinaceous stabilizing agent comprising a hyaluronic acid or a polyvinylpyrrolidone (Kollidon®), hydroxyethyl starch, alginate or a polyethylene glycol or any combination thereof, such composition being optionally pH stabilized by a suitable pH buffer, in particular by a sodium acetate buffer, or a cryoprotectant or both. Said composition may comprise in addition to the mentioned stabilizers water and at least one polyalcohol, such as mannitol or sorbitol or mixtures thereof. It may also comprise mono-, di- or higher polysaccharides, such as glucose, sucrose or fructose. Such composition is considered to be a safer composition possessing remarkable stability.

The hyaluronic acid in the instant pharmaceutical composition is in one embodiment combined with the instant neurotoxic component in a quantity of 0.1 to 10 mg, especially 1 mg hyaluronic acid per ml in a 200 U/ml botulinum toxin solution.

The polyvinylpyrrolidone (Kollidon®) when present in the instant composition, is combined with the instant neurotoxic component in such a quantity to provide a reconstituted solution comprising 10 to 500 mg, especially 100 mg polyvinylpyrrolidone per ml in a 200 U/ml neurotoxic component of botulinum toxin solution. In another embodiment reconstitution is carried out in up to 8 ml solution. This results in concentrations of down to 12.5 mg polyvinylpyrrolidone per ml in a 25 U/ml neurotoxic component solution.

The polyethyleneglycol in the instant pharmaceutical composition is in one embodiment combined with the instant neurotoxic component in a quantity of 10 to 500 mg, especially 100 mg polyethyleneglycol per ml in a 200 U/ml botulinum toxin solution. In another embodiment, the subject solution also contains a 1-100 mM, in yet another embodiment 10 mM sodium acetate buffer.

The pharmaceutical composition in accordance with the present invention in one embodiment retains its potency substantially unchanged for six month, one year, two year, three year and/or four year periods when stored at a temperature between about +8° C. and about −20° C. Additionally, the indicated pharmaceutical compositions may have a potency or percent recovery of between about 20% and about 100% upon reconstitution.

### D.2.5 Cryoprotectants

“Cryoprotectant” refers to excipients which result in an active ingredient, i.e., a neurotoxic component in a reconstituted or aqueous solution pharmaceutical composition that has greater than about 20%, 30%, 40%, 50%, 60%, 70%, 80%, 90%, and up to about 100% of the toxicity that the biologically active neurotoxic component had prior to being freeze-dried in the pharmaceutical composition.

In another embodiment, the composition may contain a polyhydroxy compound, e.g. a polyalcohol as cryoprotectant. Examples of polyalcohols that might be used include, e.g., inositol, mannitol and other non-reducing alcohols. Some embodiments of the composition do not comprise a proteinaceous stabilizer, or do not contain trehalose or maltotriose or lactose or sucrose or related sugar or carbohydrate compounds which are sometimes used as cryoprotectants.

### D.2.6 Preservatives

The terms “preservative” and “preservatives” refer to a substance or a group of substances, respectively, which prevent the growth or survival of microorganisms, insects, bacteria or other contaminating organisms within said composition. Preservatives also prevent said composition from undesired chemical changes. Preservatives which can be used in the scope of this patent are all preservatives of the state of the art known to the skilled person. Examples of preservatives that might be used include, inter alia, e.g. benzylic alcohol, benzoic acid, benzalkonium chloride, calcium propionate, sodium nitrate, sodium nitrite, sulphites (sulfur dioxide, sodium bisulfite, potassium hydrogen sulfite, etc.), disodium EDTA, formaldehyde, glutaraldehyde, diatomaceous earth, ethanol, methyl chloroisothiazolinone, butylated hydroxyanisole and/or butylated hydroxytoluene.

### D.2.7 Analgesics

The term “analgesic” relates to analgesic drugs that act in various ways on the peripheral and central nervous systems and includes inter alia Paracetamol® (acetaminophen), the nonsteroidal anti-inflammatory drugs (NSAIDs) such as the salicylates, narcotic drugs such as morphine, synthetic drugs with narcotic properties such as Tramadol®, and various others. Also included is any compound with a local analgesic effect such as e.g. lidocaine, benzylic alcohol, benzoic acid and others.

In one embodiment the analgesic is part of the composition, in another embodiment, the analgesic is administered before, during or after the treatment with the chemodenervating agent.

### D.3 Administration

As indicated above, the pharmaceutical composition comprising the botulinum toxin is administered, in one embodiment several times, in an effective amount for improving the patient's condition either prior to and/or during and/or after said locomotion therapy. In one embodiment, an effective amount of botulinum toxin is administered several times during the movement therapy, and the composition is administered for the first time before commencement of any movement/locomotion therapy.

Typically, the dose administered to the patient will be up to about 1000 units, but in general should not exceed 400 units per patient. In one embodiment the range lies between about 80 to about 400 units. These values are in one embodiment valid for adult patients. For children, the respective doses range from 25 to 800 and in another embodiment from 50 to 400 units.

While the above ranges relate to the maximum total doses, the dose range per muscle is in one embodiment within 3 to 6 units/kg body weight (b.w.), for small muscles 0.5-2 U/kg b.w., in another embodiment 0.1-1 U/kg b.w. Generally doses should not exceed 50 Upper injection site and 100 Upper muscle.

In one embodiment of the present invention the effective amount of botulinum toxin administered exceeds 500 U of neurotoxic component in adults or exceeds 15 U/kg body weight in children.

As to the frequency of dosing, the re-injection interval is in one embodiment greater than 3 months. This is particularly true when applying medicaments on the basis of the botulinum toxin complex, where there exists an increased likelihood for the occurrence of antibodies.

When applying medicaments on the basis of the neurotoxic component of botulinum toxin, such as Xeomin®, a more frequent dosing is also possible. Thus, according to the present invention the medicament to be administered is re-administered in intervals of between 3 and 6 months, in another embodiment, particularly when using the neurotoxic component of botulinum toxin, in yet another embodiment Xeomin®, the medicament is re-administered in intervals of between 2 weeks and less than 3 months. In yet another embodiment the medicament is re-administered at a point in time when muscular activity interferes with the automated movement therapy.

With regard to the composition and dosing of the medicament on the basis of botulinum toxin, and in regard to the composition, dosing and frequency of administration of the medicament on the basis of the neurotoxic component of botulinum toxin, reference is made to U.S. Ser. No. 60/817,756 incorporated herein by reference.

In one embodiment said composition comprises only botulinum toxin as an active component, in another embodiment further active components e.g. analgesics, said muscle activating agent, etc. are part of the composition.

While the above stated values are to be understood as a general guideline for administering the medicament as used within the present invention, it is, however, ultimately the physician who is responsible for the treatment who decides on both the quantity of toxin administered and the frequency of its administration. In the present method, the medicament on the basis of botulinum toxin is in one embodiment re-administered at a point in time at which the movement ability of the patient is (again) deteriorating compared to the movement ability at the point of maximum therapeutic effect of botulinum toxin.

The medicament on the basis of botulinum toxin can be to be injected directly into the affected muscles. In order to find the appropriate injection site, several means exist which help the physician in order to find the same. Within the present invention, all methods for finding the best injection site are applicable, such as injection guided by electromyography (EMG), injection guided by palpation, injection guided by CT/MRI, as well as injection guided by sonography. Among those methods, the latter is in one embodiment the method of choice when treating children. With respect to further details regarding the injection guided by sonography, we refer to Berweck “Sonography-guided injection of botulinum toxin A in children with cerebral palsy”, Neuropediatric 2002 (33), 221-223.

### D.4 The Kit

Furthermore, the invention relates to a kit for the treatment of patients suffering from movement disorders comprising a medicament comprising an effective amount of a chemodenervating agent, and a means for carrying out muscle activation therapy.

In one embodiment said means for carrying out muscle activation therapy, are one or several of the means disclosed under section B above. In one embodiment said chemodenervating agent is Botulinum toxin A. The means for muscle activation therapy can either be provided together with the chemodenervating agent or in form of an instruction leaflet.

In one embodiment, said means for carrying out muscle activation therapy is a device for carrying out automated movement therapy. Said kit comprises a driven and controlled gait orthosis and/or arm mover which guides the extremities of said patient in a physiological pattern of movement. In another embodiment it is one of the devices described in detail hereinbefore. Within the kit, the medicament in one embodiment comprises a botulinum toxin as the chemodenervating agent, in another embodiment the device is a medicament on the basis of the neurotoxic component of Botulinum toxin A.

Within said kit, the medicament is specifically adapted to be used in combination with locomotion therapy, in one embodiment in combination with the respective device that is used for said therapy. Such specific adaptation, which may be carried out specifically in relation to the kit according to the present invention but also within the medicament commercialized as such can be achieved by way of a specifically adapted packaging and/or the packaging leaflet and/or instructions of use of the medicament to be used within the present invention.

### D.5 Further Definitions

The term “lyophilization” is used in this document for a treatment of a solution containing the chemodenervating agent, e.g. the neurotoxic component of the botulinum toxin, whereas this solution is frozen and dried until only the solid components of the composition are left over. The freeze-dried product of this treatment is therefore defined in this document as “lyophilisate”.

The term “reconstitution” is defined as the process of solubilization of said freeze-dried composition of the chemodenervating agent, e.g. the neurotoxic component. This can be done by adding the appropriate amount of sterile water, e.g. if all necessary components are already contained in the lyophilisate. Or, if this is not the case, it can be done e.g. by adding a sterile saline-solution alone or if applicable with the addition of components comprising e.g. a pH buffer, excipient, cryoprotectant, preservative, analgesic stabilizer or any combination thereof. The saline of before mentioned “saline-solution” is a salt-solution, in another embodiment being a sodium-chloride (NaCl) solution, in yet another embodiment being an isotonic sodium-chloride solution (i.e. a sodium-chloride concentration of 0.9%). The solubilization is carried out in such a manner that the final “reconstitution” is directly or indirectly, i.e. for example after dilution, administrable to the patient. The neurotoxin may be reconstituted in isotonic media, e.g. in isotonic saline or in sterile saline.

The term “paresis” is defined herein under as a condition typified by partial loss of movement, or impaired movement.

### E. Remarks

With respect to the above, it is to be understood that the disclosure of all prior art (pre-published and non-prepublished) as recited above is incorporated herein by reference in full.

It is to be understood that the terminology used herein is for the purpose of describing particular embodiments only and is not intended to be limiting. It must be noted that, as used in the specification and the appended claims, the singular forms “a”, “an” and “the” include plural referents unless the context clearly dictates otherwise.

The present invention will be better understood in connection with the following examples, which are intended as an illustration of and not a limitation upon the scope of the invention.

### EXAMPLES

### Example 1

A study was carried out with 9 patients in the department of Pediatric Neurology and Developmental Medicine of the Dr. von Haunersches Children's Hospital Munich (Germany). Approval for the studies was obtained from local ethics committees.

Patients were eligible for our study if they had central gait impairment due to either congenital or acquired brain or spinal lesions. Femur length had to be at least 21 cm, which applies to children of an age of approximately 4 years. Achieving walking ability had to be a realistic goal of the rehabilitation program. Patients had to be able to reliably signal pain, fear or discomfort. Written informed consent of the parents was a prerequisite.

Exclusion criteria were: Severe lower-extremity contractures, fractures, osseous instabilities, osteoporosis, contraindication of full body load due to operations, severe disproportional bone growth, unhealed skin lesions in the lower-extremity, thromboembolic diseases, cardiovascular instability, acute or progressive neurological disorders and aggressive or self-harming behaviour.

Eight children and one adolescent were referred to our outpatient clinic for participated in the Driven Gait Orthosis (DGO) training program between January and August 2006. Mean age at the beginning of the training was 8 y 2 mo (SD2 y 10 mo, range 5 y 2 mo-14 y 4 mo). All but one patient trained on the children's module of the DGO (Table 2).

The automated locomotor training was performed by the commercially available DGO Lokomat® (Hocoma AG, Volketswil, Switzerland). The adult—as well as the new pediatric version of the DGO were used. The DGO consists of two leg orthoses which are adjustable to the anatomy of different patients. It is fastened to the legs by several braces. The width of the hip orthosis, the length of the upper and lower leg as well as the size and position of the leg braces can be varied. The main difference between the adult (femur length>350 mm) and pediatric module (210 mm to 350 mm) is in the length of the thigh. The DGO is connected to the frame of a body weight support system by a four bar linkage. This allows movement of the orthosis in a vertical direction and provides additional vertical stability. On each leg, two linear drives move the hip and the knee joint of the orthosis. These drives are controlled by a position controller (real-time system implemented on a PC) that conducts a kinematic pattern resembling normal walking. The movements of the DGO are synchronized with the treadmill. The walking speed can be set between 1 and 3.2 km/h. To ensure safe training, several safety features are integrated into the system. These include stop buttons for the therapists and the patients and a controller that limits both excessive forces at the drives and deviations from the desired position in the joint angles. The dorsiflexion of the ankle joint is provided by an elastic foot strap. To ensure a most physiological gait pattern and to prevent the skin from excoriation, proper fixation of the patient to the DGO is of utmost importance. For body weight support, a counter weight system is used. This allows body weight support within a range of 5 to 80 kg in 5 kg steps.

The amount of unloading was set at 50 percent of body weight initially, to be decreased successively according to the gain of muscular strength (allowing no excessive knee flexion during stance). The initial gait velocity for the training was chosen according to the capabilities of the child.

Training on the DGO included three to four sessions of 2545 minutes per week. 10 to 13 sessions were conducted. Nearly all of the patients stopped their usual weekly physiotherapy sessions because of time limits. Eight out of 10 patients were referred for Botox® treatment of muscles of the lower extremity (Table 2).

To assess the feasibility of the DGO training, walking time, covered distance and gait speed of each session were logged. Motor performance tests were assessed before and at the end of the training program. Gait speed was assessed with the 10 Meter Walking Test (10MWT). Children were instructed to walk at their comfortable speed.

To assess changes in motor functions, the standing (dimension D) and walking sections (dimension E) of the GMFM-66 were administered by a GMFM (Gross Motor Function Measure)-certificated therapist. Additionally, children of the inpatient group performed a 6-Min Walking Test (6MWT) to evaluate gait endurance. To determine the amount of assistance the child requires during walking, the Functional Ambulation Categories (FAC) were used. All tests were accomplished using the same assistive devices before and after the intervention.

Parameters were checked in regard to their respective normal distribution. This applied to the results of gait speed and 6MWT. Thus differences between pre- and posttraining were analyzed using the repeated t-test. All other parameters were analyzed with the nonparametric Wilcoxon signed rank test.

Eight of the nine patients completed the training on the DGO. One patient dropped out after the third training due to reduced compliance. The total number of training sessions on the DGO was 12 (SD 1.0, range 10-13). The participants walked on average 1158 m (SD 371 m, range 410-1675 m) per session. Mean training duration was 28:42 minutes (SD 3:30 minutes, range 23-32 minutes). The average walking speed was 1.7 km/h (SD 0.17 km/h, range 1.5-2.1 km/h) with unloading of 14.4% (SD 12.6% of body weight, range 0-30%) (FIG. 2c,d).

Over-ground walking parameters were assessed in seven patients and improved in all of them. Mean gait speed increased significantly from 0.87 m/s (SD 0.32 m/s) to 1.09 m/s (SD 0.31 m/s, T=−3.11, p=0.01) (FIG. 3e). Seven of the eight patients showed an improvement in dimension D and E. The mean score of dimension D changed markedly from 46.7 (SD 34.1) to 52.0 (SD 28.8), although statistical analysis revealed no significance (Z=−1.820, p=0.69). In dimension E the mean score increased slightly but significantly from 39.5 (SD 32.6) to 42.2 (SD 34.6) after training (Z=−2,366, p<0.05). (FIG. 3f+g) Walking abilities, as assessed with FAC, showed no changes.

Gait speed and the GMFM score improved significantly. Results of the walking section in the GMFM revealed significant improvements in contrast to the less distinct findings in the standing section. An overview of the study is given in Table 2 below.

TABLE 2

| | | | | | | Total | |
|---|---|---|---|---|---|---|---|
| | | | | | Number | walking | BoNT/A |
| Patient | | Age | | Lokomat- | of | distance | Treatment |
| No. | Sex | (y:m) | Diagnosis | Type | trainings | (km) | before |
| 1 | F | 6:4 | CP (I) | CM | 13 | 17.2 | Y |
| 2 | F | 9:10 | CP (II) | CM | 12 | 13.6 | Y |
| 3 | F | 10:10 | CP (II) | CM | 13 | 18.6 | N |
| 4 | M | 6:2 | CP (III) | CM | 10 | 9.9 | Y |
| 5 | M | 6:4 | CP (III) | CM | 12 | 13.3 | Y |
| 6 | M | 14:4 | CP (II) | AM | 12 | 12.5 | Y |
| 7 | F | 7:0 | CP (IV) | CM | 3 | 0.9 | Y |
| 8 | M | 8:0 | CP (IV) | CM | 10 | 5.0 | N/Y |
| 9 | M | 5:2 | CP (III) | CM | 10 | 6.7 | Y |

### Example 2

An 8 year old male patient with a bilateral spastic cerebral palsy due to prematurity associated brain damage was subjected to 12 sessions of Robotic assisted treadmill training using the Pediatric Lokomat®. The patient was not able to perform more than 2 training session as the internal control of the robotic device stopped the training due to elevated resistance related to increased muscle tone. Botulinum toxin treatment (total dose 15 U/kg Botox®) was done in a multilevel approach of the lower extremity including hip flexors, knee flexors, adductor muscles and gastrocnemius muscle. After this intervention the Robotic assisted treadmill training was easily continued and the patient was able to perform all suggested 12 sessions. After these interventions there were significant improvements of endurance (from 344-751 m in the 6 min running test) and motor function (from 2.5% to 10% in walking dimension of the Gross Motor Function measure).

This example shows the synergetic effect of combining the administration of a denervating agent such as botulinum toxin leading to an at least temporary relief of muscle spasticity with movement therapy that can only effectively work on an at least partially relaxed muscle. A muscle then strengthened or conditioned by the movement therapy may be exposed to a potentially higher dose of denervating agent, thus leading to further synergetic effects.

### Example 3

An 25 year old patient suffering from torticollis spasmodicus (cervical dystonia) is injected between 0.1-1000 units/kg body weight of the neurotoxic component of Botulinum toxin A before, during and after a muscle activation therapy comprising alternating cycles of heating and cooling of the sternocleidomastoid muscle of the neck. After this intervention there is a significant improvement according to the Toronto Western Spasmodic Torticollis Rating Scale (TWSTRS).

### Example 4

An 44 year old patient suffering from torticollis spasmodicus (cervical dystonia) is injected between 0.1-1000 units/kg body weight of the neurotoxic component of Botulinum toxin A before, during and after a muscle activation therapy comprising repeated electrical stimulation of the sternocleidomastoid muscle of the neck. After this intervention there is a significant improvement according to the Toronto Western Spasmodic Torticollis Rating Scale (TWSTRS).

### Example 5

An 24 year old patient suffering from torticollis spasmodicus (cervical dystonia) is injected between 0.1-1000 units/kg body weight of the neurotoxic component of Botulinum toxin A before, during and after a muscle activation therapy comprising placing said patient on a platform vibrating between 12 Hz and 20 Hz. After this intervention there is a significant improvement according to the Toronto Western Spasmodic Torticollis Rating Scale (TWSTRS).

### Example 6

An 37 year old patient suffering from torticollis spasmodicus (cervical dystonia) is injected between 0.1-1000 units/kg body weight of the neurotoxic component of Botulinum toxin A before, during and after a muscle activation therapy comprising the application of ultrasonic sound waves of 40 kHz to the sternocleidomastoid muscle of the neck. After this intervention there is a significant improvement according to the Toronto Western Spasmodic Torticollis Rating Scale (TWSTRS).

### Example 7

An 34 year old patient suffering from torticollis spasmodicus (cervical dystonia) is injected between 0.1-1000 units/kg body weight of the neurotoxic component of Botulinum toxin A before, during and after a muscle activation therapy comprising hydrostatic massage of the sternocleidomastoid muscle of the neck. After this intervention there is a significant improvement according to the Toronto Western Spasmodic Torticollis Rating Scale (TWSTRS).

### Example 8

An 43 year old patient suffering from torticollis spasmodicus (cervical dystonia) is injected between 0.1-1000 units/kg body weight of the neurotoxic component of Botulinum toxin A before, during and after a muscle activation therapy comprising the application of therapeutic microwaves of low intensity to the sternocleidomastoid muscle of the neck. After this intervention there is a significant improvement according to the Toronto Western Spasmodic Torticollis Rating Scale (TWSTRS).

### Example 9

An 49 year old patient suffering from torticollis spasmodicus (cervical dystonia) is injected between 0.1-1000 units/kg body weight of the neurotoxic component of Botulinum toxin A before, during and after a muscle activation therapy comprising the application of a saponin (DSS) isolated from the root of Dalbergia saxatilis to the sternocleidomastoid muscle of the neck. After this intervention there is a significant improvement according to the Toronto Western Spasmodic Torticollis Rating Scale (TWSTRS).

### Example 10

A 33 year old patient suffering from torticollis spasmodicus (cervical dystonia) is injected between 0.1-1000 units/kg body weight of the neurotoxic component of Botulinum toxin A before, during and after a muscle activation therapy comprising the application of the casein kinase I to the sternocleidomastoid muscle of the neck. After this intervention there is a significant improvement according to the Toronto Western Spasmodic Torticollis Rating Scale (TWSTRS).

### Example 11

A 56 year old patient suffering from torticollis spasmodicus (cervical dystonia) is injected between 0.1-1000 units/kg body weight of the neurotoxic component of Botulinum toxin A before, during and after a muscle activation therapy comprising the application potassium (K⁺) to the sternocleidomastoid muscle of the neck. After this intervention there is a significant improvement according to the Toronto Western Spasmodic Torticollis Rating Scale (TWSTRS).

1. A chemodenervating agent which is administered to a patient in an effective amount to treat a movement disorder in the patient, wherein the patient is a patient who is, has been and/or will be subjected to a muscle stimulation therapy, and wherein the chemodenervating agent is administered prior to and/or during and/or after the muscle stimulation therapy.

2. The chemodenervating agent of claim 1, wherein the chemodenervating agent is a botulinum toxin.

3. The chemodenervating agent of claim 2, wherein the botulinum toxin is selected from the group consisting of serotypes A, B, C, D, E, F, G and a mixture thereof.

4. The chemodenervating agent of claim 3, wherein the botulinum toxin is a botulinum toxin complex type A.

5. The chemodenervating agent of claim 2, wherein the botulinum toxin is a neurotoxic component of a Clostridium botulinum toxin complex.

6. The chemodenervating agent of claim 5, wherein the neurotoxic component is of type A.

7. The chemodenervating agent of claim 2, wherein the effective amount of botulinum toxin administered exceeds 500 U of neurotoxic component in adults or exceeds 15 U/kg body weight in children.

8. The chemodenervating agent of claim 1, wherein the muscle stimulation therapy is an automated muscle stimulation therapy.

9. The chemodenervating agent of claim 1, wherein the muscle stimulation therapy is a muscle activation therapy, wherein the muscle activation refers to an elevation of muscle metabolism above resting state of the muscle.

10. The chemodenervating agent of claim 1, wherein the muscle stimulation therapy is an automated movement therapy.

11. A method of treating a movement disorder in a patient, the method comprising administering a composition comprising an effective amount of a chemodenervating agent to the patient, wherein the patient is a patient who is, has been and/or will be subjected to a muscle stimulation therapy, and wherein the chemodenervating agent is administered prior to and/or during and/or after the muscle stimulation therapy.

12. The method of claim 11, wherein the muscle stimulation therapy is an automated muscle stimulation therapy.

13. The method of claim 11, wherein the muscle stimulation therapy is a muscle activation therapy, wherein the muscle activation is an elevation of muscle metabolism above resting state of the muscle.

14. The method of claim 12, wherein the muscle stimulation therapy is an automated movement therapy.

15. The method of claim 13, wherein the muscle activation therapy is temperature stimulation, electric stimulation, vibration, activation by sound-waves, activation by hydrostatic means, activation by electro-magnetic waves or magnetic fields, pharmaceutical activation or any combination thereof.

16. The method of claim 15, wherein the temperature stimulation is a heating of the target muscle above 40°, or above 45° C., or above 50° C., up to 55° C., up to 60° C., up to 70° C. or up to 80° C.

17. The method of claim 15, wherein the automated muscle activation by temperature stimulation is a cooling of the target muscle to below 35° C., or below 30° C., or below 25° C., or below 20° C., or below 10° C., down to 0° C., down to −5° C., down to −10° C. or down to −20° C.

18. The method of claim 15, wherein the electric stimulation is directed to the nerves innervating the target muscle.

19. The method of claim 15, wherein the electric stimulation is directed to the target muscle itself.

20. The method of claim 15, wherein the vibration is directed to the whole body.

21. The method of claim 15, wherein the vibration is directed to a single muscle, muscle group or limb.

22. The method of claim 15, wherein the sound-waves are ultrasound waves or acoustical waves.

23. The method of claim 15, wherein the hydrostatic means comprise water-jets.

24. The method of claim 15, wherein the electro-magnetic waves comprise microwaves.

25. The method of claim 15, wherein the magnetic fields comprise magnetic stimulation.

26. The method of claim 15, wherein the pharmaceutical activation comprises the administration of a stimulant, a muscle contractant, a substance which increases blood flow within the muscle, a substance which raises the muscle temperature, a substance which up-regulates the number of surface proteins thereby allowing the chemodenervating agent to bind and enter the cell or any combination thereof.

27. The method of claim 26, wherein the stimulant is selected from the group of a β₃ agonist, caffeine, ephedrine, amphetamine, methamphetamine, methylphenidate, cocaine-derivate and any combination thereof.

28. The method of claim 26, wherein the muscle contractant is selected from the group of a substance with sympathetic effect, a substance with agonistic effects on β₂-adrenergic receptor, caffeine, acetylcholine, nicotine, epibatidine-derivatives, ABT-594, dimethylphenylpiperazinium, succinyl choline, a muscle stimulating saponin-derivative isolated from Dalbergia saxatilis, calcium, potassium, norepinephrine, adrenaline (epinephrine), leukotrienes, allene containing arachidonic acid derivatives and any combination thereof.

29. The method of claim 26, wherein the substance which increases blood flow within the muscle is selected from the group of EDHF, interstitial K⁺, nitric oxide, β₂ adrenergic agonists, histamine, prostacyclin, prostaglandin, VIP, extracellular adenosine, extracellular ATP, extracellular ADP, L-Arginine, bradykinin, substance P, niacin (nicotinic acid), platelet activating factor (PAF), CO₂, interstitial lactic acid, Adenocard®, alpha blockers, amyl nitrite, atrial natriuretic peptide, ethanol, histamine-inducers, complement proteins C3a, C4a, C5a, nitric oxide inducers, glyceryl trinitrate (nitroglycerin), isosorbide mononitrate, isosorbide dinitrate, pentaerythritol tetranitrate (PETN), sodium nitroprusside, PDE5 inhibitors, agents which indirectly increase the effects of nitric oxide, sildenafil, tadalafil, tardenafil, tetrahydrocannabinol, theobromine, papaverine and any combination thereof.

30. The method of claim 26, wherein the substance which raises the muscle temperature is selected from the group of ephedra, bitter orange (synephrine), capsicum, ginger, sibutramine and its metabolites, caffeine and any combination thereof.

31. The method of claim 26, wherein the surface protein is selected from the group comprising a substance which up-regulates SV2, GT1b, GD1b, GQ1b, synaptotagmin polypeptides, Syt1 and Syt2.

32. The method of claim 26, wherein the substance which up-regulates the number of surface proteins is selected from the group comprising hormones, growth factors, neurotrophins, blocking substances of receptor-internalization, factors which enhance the receptor surface expression, arrestin-inhibitors, protease inhibitors, blocking substances of receptor degradation, inhibitors of inhibitory G-proteins, competitive receptor antagonists and neurotransmitter degrading agents.

33. The method of claim 14, wherein the automated movement therapy is supported by an automated gait orthosis or an arm mover.

34. The method of claim 33, wherein the automated gait orthosis is used in combination with a treadmill.

35. The method of claim 14, wherein the automated movement therapy is carried out by using a device comprising a driven and controlled orthotetic device which guides the legs of the patient in a physiological pattern of movement, in one embodiment using a treadmill and a relief mechanism acting on the body weight of the patient.

36. The method of claim 35, wherein the relief mechanism comprises means for adjusting the height of and the relief force acting on the weight of the patient, wherein the weight is supported by a cable, with a first cable length adjustment means to provide an adjustment of the length of the cable to define the height of the suspended weight and a second cable length adjustment means to provide an adjustment of the length of the cable to define the relief force acting on the suspended weight.

37. The method of claim 35, wherein the automated movement therapy is carried out by employing an apparatus for treadmill training, comprising a treadmill, a relief mechanism for the patient, and a driven orthotic device, wherein a parallelogram fixed in a height-adjustable manner on the treadmill is provided for stabilizing the orthotic device and preventing the patient from tipping forward, backwards and sidewards, the parallelogram being attached to the orthotic device; the orthotic device comprises a hip orthotic device and two leg parts, whereby two hip drives are provided for moving the hip orthotic device, and two knee drives are provided for moving the leg parts; the hip orthotic device and leg parts are adjustable, the leg parts are provided with cuffs which are adjustable in size and position; a control unit is provided for controlling the movements of the orthotic device and controlling the speed of the treadmill.

38. The method of claim 35, wherein the automated movement therapy is carried out by employing an apparatus for treadmill training, comprising a treadmill including a railing, a relief mechanism for the patient, and a driven orthotic device, wherein means for stabilizing the orthotic device are provided that prevent the patient from tipping forward, backward and sideward; the orthotic device comprises a hip orthotic device and two leg parts, two hip drives are provided for moving the hip orthotic device, and two knee drives are provided for moving the leg parts; a ball screw spindle drive is provided for each knee drive and hip drive, the orthotic device and leg parts are adjustable, the leg parts are provided with cuffs which are adjustable in size and position; a control unit is provided for controlling the movements of the orthotic device and controlling the speed of the treadmill.

39. The method of claim 14, wherein the automated movement therapy is carried out by employing an apparatus for locomotion therapy for the rehabilitation or habilitation of bilateral or unilateral spastic conditions in paraparetic and hemiparetic patients, comprising a standing table adjustable in height and inclination, a fastening belt with holding devices on the standing table for the patient, a drive mechanism for the leg movement of the patient, consisting of a knee mechanism and a foot mechanism, wherein the standing table has a head portion displaceable with respect to a leg portion about a pivot joint, whereby the pivot joint provides an adjustable hip extension angle for which an adjusting mechanism is provided; and the knee portion and foot portion are displaceably arranged on rails on the leg mechanism; the foot mechanism serves to establish force on the sole of the foot during knee extension; a control unit is provided for controlling movement of the apparatus.

40. The method of claim 14, wherein the automated movement therapy is carried out by employing a device for applying a force between first and second portions of an animate body, the device comprising: first and second link assemblies associated with the first and second portions, respectively, each the first and second link assembly comprising:
a) a supporting section secured in position on a portion, each supporting section being a supporting link; and
b) an articulated link attached through a joint to each of the supporting links; wherein the articulated links of the first and second link assemblies are attached to each other through a pivot joint, with the articulated link of the second assembly extending beyond the pivot joint; first and second casings attached to a link in the first link assembly; first and second tendons extending through the first and second casings, respectively, and attached to a link in the second link assembly, wherein one of the tendons is attached to the articulated link in the second link assembly on one side of the pivot joint and the other tendon is attached to the articulated link in the second link assembly on the opposite side of the pivot joint.

41. The method of claim 14, wherein the automated movement therapy is carried out by employing a device for applying a force between first and second portions of a hand, one of the portions being a phalanx, the device comprising: first and second link assemblies associated with the first and second portions, respectively, each link assembly comprising:
a) a supporting section secured in position on a portion, each supporting section being a supporting link; and
b) an articulated link attached through a joint to each of the supporting links; wherein the articulated links of the first and second link assemblies are attached to each other through a pivot joint, with the articulated link of the second assembly extending beyond the pivot joint; first and second casings attached to a link in the first link assembly; and first and second tendons extending through the first and second casings, respectively, and attached to a link in the second link assembly, wherein one of the tendons is attached to the articulated link in the second link assembly on one side of the pivot joint and the other tendon is attached to the articulated link in the second assembly on the opposite side of the pivot joint.

42. The method of claim 11, wherein the movement disorder is a hyperkinetic and/or hypokinetic movement disorder, wherein an imbalance between agonist and antagonist is interfering with function.

43. The method of claim 42, wherein the movement disorder is associated with cerebral palsy, M. Parkinson, central gait impairment, spinal cord injuries, dystonias, traumatic brain injury, genetic disorders, metabolic disorders, dynamic muscle contractures and/or stroke.

44. The method of claim 43, wherein the movement disorder is associated with at least one selected among pes equinus, pes varus, lower limb spasticity, upper limb spasticity, adductor spasticity, hip flexion contracture, hip adduction, knee flexion spasticity (crouch gait), plantar flexion of the ankle, supination and pronation of the subtalar joint, writer's cramp, musician's cramp, golfer's cramp, leg dystonia, thigh adduction, thigh abduction, knee flexion, knee extention, equinovarus deformity, foot dystonia, striatal toe, toe flexion, toe extension.

45. The method of claim 11, wherein the patient is human.

46. The method of claim 45, wherein the patient has not completed its motor development and fixed muscle contractures have not occurred.

47. The method of claim 46, wherein the patient is a child up to six years in age.

48. The method of claim 11, wherein the chemodenervating agent is a botulinum toxin.

49. The method of claim 11, wherein the chemodenervating agent is administered by injection.

50. The method of claim 11, wherein the chemodenervating agent is administered several times during the treatment.

51. The method of claim 11, wherein the chemodenervating agent is administered for the first time before commencement of the movement therapy.

52. The method of claim 11, wherein the chemodenervating agent is re-administered in intervals of between 3 and 6 months.

53. The method of claim 11, wherein the chemodenervating agent is re-administered in intervals of between 2 weeks and less than 3 months.

54. The method of claim 11, wherein the chemodenervating agent is re-administered at a point in time when muscular activity interferes with the automated muscle activation therapy.

55. The method of claim 48, wherein the effective amount of botulinum toxin administered exceeds 500 U of neurotoxic component in adults or exceeds 15 U/kg body weight in children.

56. The method of claim 48, wherein the botulinum toxin is a botulinum toxin complex type A.

57. The method of claim 57, wherein the botulinum toxin is selected from the group consisting of serotypes A, B, C, D, E, F, G and a mixture thereof.

58. The method of claim 48, wherein the botulinum toxin is a neurotoxic component of a Clostridium botulinum toxin complex.

59. The method of claim 58, wherein the neurotoxic component is of type A.

60. A kit for the treatment of patients suffering from movement disorders comprising,
a) a medicament comprising an effective amount of a chemodenervating agent; and
b) means for carrying out a muscle stimulation therapy.

61. The kit of claim 60, wherein the means for carrying out the muscle stimulation therapy is selected from the group of temperature stimulation means, electric stimulation means, vibration means, activation by sound-wave means, hydrostatic means, electro-magnetic wave means and magnetic field means, or any combination thereof.

62. The kit of claim 60, wherein the means for carrying out the muscle stimulation therapy is an automated movement therapy comprising a driven and controlled gait orthosis and/or arm mover which guides the extremities of the patient in a physiological pattern of movement.

63. The kit of claim 60, wherein the chemodenervating agent is a botulinum toxin.

64. The kit of claim 63, wherein the botulinum toxin is a neurotoxic component of a Clostridium botulinum toxin complex.
